(19) United States
(12) Patent Application Publication
(10) Pub. No.: US 2026/0174735 A1
(43) Pub. Date: Jun. 25, 2026

(54) STABLE ORAL PHARMACEUTICAL FORMULATIONS

(71) Applicant: Method Pharmaceuticals, LLC, Southlake, TX (US)

(72) Inventors: Jitesh Devendra, Chennai (IN); Sampathkumar Devarajan, Chennai (IN); Mathivanan Rajagopal, Chennai (IN); Bindumadavan Kannan, Chennai (IN); Ramesh Subramani, Chennai (IN); Jeevanantham Raji, Chennai (IN); Matthew Scott Tucker, Southlake, TX (US); Christopher Boone, Southlake, TX (US)

(21) Appl. No.: 19/430,629

(22) Filed: Dec. 23, 2025

Related U.S. Application Data
(60) Provisional application No. 63/738,404, filed on Dec. 23, 2024.

(51) Int. Cl. A61K 31/401; A61K 9/00; A61K 31/549; A61K 31/55; A61K 47/12; A61K 47/26; A61K 47/32; A61K 47/34; A61K 47/38; A61K 47/40

(52) U.S. Cl. CPC A61K 31/401; A61K 9/0053; A61K 31/549; A61K 31/55; A61K 47/12; A61K 47/26; A61K 47/32; A61K 47/34; A61K 47/38; A61K 47/40

(57) ABSTRACT

The present disclosure relates to pharmaceutical formulations of angiotensin converting enzyme inhibitor and/or diuretic, particularly stable oral pharmaceutical formulations comprising angiotensin converting enzyme inhibitor and/or diuretic with one or more pharmaceutically acceptable excipients for the treatment of hypertension, coronary artery disease, heart failure, acute myocardial infarction and other related cardiovascular diseases.

### CROSS-REFERENCE TO RELATED APPLICATIONS

This application claims the benefit of, and priority to, U.S. Provisional Patent Application No. 63/738,404, filed on Dec. 23, 2024, the entire contents of which is hereby incorporated herein by reference.

### FIELD OF THE INVENTION

The present disclosure provides stable oral liquid pharmaceutical formulation comprising angiotensin converting enzyme inhibitor and/or diuretic with one or more pharmaceutically acceptable excipients for the treatment of hypertension, coronary artery disease, heart failure, acute myocardial infarction and other related cardiovascular diseases.

### BACKGROUND OF THE INVENTION

Hypertension, or high blood pressure, is a serious health issue in many countries. Left unchecked, hypertension is considered a substantial risk factor for cardiovascular and other related diseases including coronary heart disease, myocardial infarction, congestive heart failure, stroke and kidney failure. Hypertension is classified as primary hypertension or secondary hypertension. Primary hypertension has no known cause and may be related to a number of environmental, lifestyle and genetic factors such as stress, obesity, smoking, inactivity and sodium intake. Secondary hypertension can be caused by drug or surgical interventions or by abnormalities in the renal, cardiovascular or endocrine system.

Many blood pressure medications, known as antihypertensives, are available by prescription to lower high blood pressure or hypertension. Various therapeutic classes of drugs are available and includes alpha-adrenergic blockers, beta-adrenergic blockers, calcium channel blockers, mineralocorticoid antagonists, central alpha-agonists, diuretics and rennin-angiotensin-aldosterone inhibitors, which include angiotensin II receptor antagonists and angiotensin converting enzyme inhibitors. Angiotensin converting enzyme inhibitors inhibit angiotensin converting enzyme, a peptidyl-dipeptidase that catalyzes angiotensin I to angiotensin II, a potent vasoconstrictor involved in regulating blood pressure.

It is currently known that angiotensin converting enzyme is an enzyme with multiple actions, that is, it acts in several substrates. Apart from acting as a dipeptidase in angiotensin I and in bradykinin, it is also capable of cleaving other peptides, indicating that the enzyme may act in several tissues and systems. An important advantage of angiotensin converting enzyme inhibitors is to prevent the harmful effects of angiotensin II in cardiac and vascular remodeling, being the antihypertensive class of highest efficacy in reducing left ventricular hypertrophy and vascular stiffness, also improving endothelial dysfunction. They are useful to heart failure patients with or without associated hypertension, also improving heart failure survival. So, they reduce cardiac remodeling in systolic heart failure and in post-acute myocardial infarction.

Angiotensin converting enzyme inhibitors are generally very difficult to formulate into dosage forms as certain angiotensin converting enzyme inhibitors upon contact with commonly used pharmaceutically acceptable excipients are prone to various types of degradation such as cyclization via internal nucleophilic attack to form substituted diketopiperazines, hydrolysis of the side-chain ester group and oxidation to form products having often unwanted coloration.

In many of these conditions, patients may, as a result of the conditions, or as a result of comorbidities, have difficulty in taking a drug, or be unwilling or unable to take a drug in tablet or capsule form that leads to lack of dose maintenance ant it may also result in an incorrect dosage amount, and thus, may be problematic for the treatment of a patient with hypertension and/or coronary artery disease till their life term, at least because a satisfactory therapeutic effect may not be achieved.

The oral liquid dosage form addresses patients that have difficulty in swallowing tablets. Liquids are easier to swallow than solid dosage forms and provides more rapid absorption leading to faster therapeutic response. Oral liquids offer dosing flexibility and can be easily administered to geriatric, neonatal and pediatric patients.

Formulating as an oral liquid formulation, gives the advantage of dose titration for accurate dose, ease of administration from pediatric to geriatric patients. The oral liquid formulations provide clinicians and patients with convenient and palatable alternative dosing formats that allow precise adjustment of dose and enhance patient compliance with clinician orders.

While investigating oral solution/suspension dosage forms, it was discovered that stability depends on the inactive ingredients present in the formulation. Oral liquid formulation may assist with better bioavailability because the drug is already distributed in solution/suspension dosage form, which provides a homogenous system with less chance of dose variation.

Most of the angiotensin converting enzyme inhibitors are generally unpalatable and have a bitter and/or metallic taste or after taste, have a high degree of patient non-compliance, making taste-masking an important area of pharmaceutical research. Several taste-masking approaches and technologies have been used and reviewed such as addition of flavoring agents, addition of sweetening agents, microencapsulation, ion-exchange, inclusion complexation, granulation, adsorption, pro-drug development, addition of bitterness inhibitors, emulsion techniques, pH modification, gelation or enhanced viscosity, liposome entrapment, and addition of effervescent agents.

Flavorings and sweeteners are not appropriate for solitary use in preparations of very bitter tasting drugs, as addition of a flavor or sweetening agent alone is insufficient at masking the taste of a very bitter drug. The choice of taste-masking strategy depends on several factors, such as, for example, the extent of the bitter taste of the API, dose levels, solubility and ionic characteristics, desired bioavailability, and required dosage form. Accordingly, the practice of taste masking APIs is highly unpredictable. Typically, determining an effective taste-masking formulation requires extensive experimentation, and a skilled person cannot readily predict which taste masking strategy, or combinations thereof, will be suitable.

It has been recognized that monotherapy achieve less blood pressure goals in the majority of patients with hypertension, particularly those with stage two hypertension and those with comorbidities, such as diabetes, high coronary disease risk profile, heart failure and/or a history of myocardial infarction. Most patients require two or more antihypertensive agents from complementary classes to achieve blood pressure control.

The blood pressure lowering ability of an angiotensin converting enzyme inhibitor is enhanced by the administration of a diuretic, particularly in patients with a salt-sensitive form of hypertension. This type of response has been the basis for the development of fixed dose combination products consisting of an angiotensin converting enzyme inhibitor and a thiazide diuretic. The underlying principle for combining these two drug classes is that diuretic induced sodium depletion activates the renin-angiotensin axis and moves blood pressure to an angiotensin-II-dependent mode.

Despite extensive clinical experiences on fixed dose combination of an angiotensin converting enzyme inhibitor with other diuretic, Hydrochlorothiazide remains the more popular diuretic choice among clinicians in the context of developing antihypertensive combinations.

United States Food and Drug Administration Agency's approved fixed dose combination drugs consisting of hydrochlorothiazide with angiotensin converting enzyme inhibitors include Benazepril, Captopril, Enalapril, Fosinopril, Lisinopril, Quinapril or Moexipril.

Above approved drugs are related to oral solid dosage forms, in particular, specific to tablets only and there is no commercial granted product available in oral liquid dosage form of an angiotensin converting enzyme inhibitor and/or a diuretic with other pharmaceutically acceptable excipients.

Accordingly, there is a need for developing oral liquid pharmaceutical products comprising of an angiotensin converting enzyme inhibitor and/or a diuretic with other pharmaceutically acceptable excipients and the stable oral liquid pharmaceutical formulations of the current disclosure disclosed herein serve to solve the above-mentioned drawbacks.

Therefore, described herein are the stable oral liquid pharmaceutical products comprising of angiotensin converting enzyme inhibitor and/or diuretic with other pharmaceutically acceptable excipients.

### SUMMARY OF THE INVENTION

The present disclosure relates to manufacturable and simultaneously storage-stable and bioavailable oral liquid formulations of angiotensin converting enzyme inhibitor and/or diuretic with one or more pharmaceutically acceptable excipients for cardiovascular and other related diseases. Further, stable oral liquid pharmaceutical formulations of angiotensin converting enzyme inhibitor and/or diuretic provide methods of treating hypertension, heart failure, acute myocardial infarction, comprising administering to a patient in need thereof.

In aspects, a stable oral pharmaceutical formulation is provided including at least one of Lisinopril or Benazepril with Hydrochlorothiazide, Benazepril, Hydrochlorothiazide, a pharmaceutically acceptable salt thereof, a hydrate thereof, a solvate thereof, and/or combinations thereof, and a sweetening agent.

In further aspects, a stable oral pharmaceutical formulation is provided including an angiotensin converting enzyme inhibitor and/or a diuretic; a sweetening agent; and a pharmaceutically acceptable excipient, wherein the angiotensin converting enzyme inhibitor is in an amount of from about 0.1% to about 2% w/w, wherein the diuretic is in an amount of from about 0.15% to about 2.6% w/w, and wherein the sweeting agent is in an amount of from about 0.5% to about 70% w/w.

In other aspects, methods of treating a patient in need of therapy for hypertension, coronary artery disease, heart failure, acute myocardial infarction and other related cardiovascular diseases, are provided which include administering a therapeutically effective amount of the stable oral pharmaceutical formulations of the present disclosure.

### DETAILED DESCRIPTION OF THE INVENTION

The following paragraphs detail various embodiments of the invention. For the avoidance of doubt, it is specifically intended that any particular feature(s) described individually in any one of these paragraphs (or part thereof) may be combined with one or more other features described in one or more of the remaining paragraphs (or part thereof). In other words, it is explicitly intended that the features described below individually in each paragraph (or part thereof) represent important aspects of the invention that may be taken in isolation and combined with other important aspects of the invention described elsewhere within this specification as a whole and including the examples and figures. The skilled person will appreciate that the invention extends to such combinations of features and that these have not been recited in detail here in the interests of brevity.

In the context of describing the oral liquid pharmaceutical formulation described herein and in the appended claims, the singular forms “a”, “an”, and “the” include plural references unless the context clearly dictates otherwise. Thus, for example, reference to “an excipient” is a reference to one or more excipients and equivalents thereof which will become apparent to those persons skilled in the art upon reading this disclosure and so forth.

In the context of describing the oral liquid pharmaceutical formulation described herein and in the appended claims, a reference to “a method” or “a process” includes one or more methods, one or more processes and/or steps of the type described herein and/or which will become apparent to those persons skilled in the art upon reading this disclosure and so forth.

The term “about” is used to indicate that a value includes the standard level of error for the device or method being employed to determine the value. As used herein, the term “about” means a slight variation of the value specified, for example, preferably within 10% of the value specified. The use of the term “or” in the claims is used to mean “and/or” unless explicitly indicated to refer to alternatives only or the alternatives are mutually exclusive, although the disclosure supports a definition that refers to only alternatives and to “and/or.” The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps; and also covers other unlisted steps.

The expressions “optional” or “optionally” means that the subsequently described event or circumstance may or may not occur, and that the description includes instances where the event occurs and instances where it does not.

The term “therapeutically effective amount” or “effective dose” as used herein refers to the amount or dose of pharmaceutically active ingredient that is sufficient to initiate therapeutic response in a patient.

The expression “pharmaceutically acceptable,” as used herein refers to an excipient having compatibility with the other ingredients of the formulation and not deleterious to the recipient thereof and also it means it is useful in preparing a pharmaceutical formulation that is generally safe and non-toxic.

The expression “pharmaceutically acceptable salts” include conventionally used non-toxic salts, for example a salt with an inorganic base such as an alkali metal salt (such as sodium salt and potassium salt), an alkaline earth metal salt (such as calcium salt and magnesium salt), an ammonium salt; or a salt with an organic base, for example, an amine salt (such as methylamine salt, dimethylamine salt, cyclohexylamine salt, benzylamine salt, piperidine salt, ethylenediamine salt, ethanolamine salt, diethanolamine salt, triethanolamine salt, tris(hydroxymethylamino) ethane salt, monomethyl-monoethanolamine salt, procaine salt and caffeine salt), a basic amino acid salt (such as arginine salt and lysine salt), tetraalkyl ammonium salt and the like, or other salt forms that enable the pulmonary hypertension reducing agent to remain soluble in a liquid medium, or to be prepared and/or effectively administered in a liquid medium, including an aqueous medium. The above salts may be prepared by a conventional process, for example from the corresponding acid and base or by salt interchange.

For example, the hypertension reducing agent may be employed in a free base form or in a salt form (e.g., as pharmaceutically acceptable salts). Examples of suitable pharmaceutically acceptable salts include inorganic acid addition salts such as hydrochloride, hydrobromide, sulfate, phosphate, and nitrate; organic acid addition salts such as acetate, propionate, succinate, lactate, glycolate, malate, tartrate, citrate, maleate, fumarate, methansulfonate, p-toluenesulfonate, and ascorbate; salts with acidic amino acid such as aspartate and glutamate; alkali metal salts such as sodium salt and potassium salt; alkaline earth metal salts such as magnesium salt and calcium salt; ammonium salt; organic basic salts such as trimethylamine salt, triethylamine salt, pyridine salt, picoline salt, dicyclohexylamine salt, and N,N′-dibenzylethylenediamine salt; and salts with basic amino acid such as lysine salt and arginine salt. The salts may be in some cases hydrates or ethanol solvates.

The term “excipient” means a pharmacologically inactive component such as sweetening agent, complexing agent, stabilizing agent, viscosity modifiers, flavoring agent, preservative, buffering agent, suspending agent, solvent, co-solvent, chelating agent, suspension aids, etc., of a pharmaceutical product. The excipients that are useful in preparing a pharmaceutical formulation are generally safe, non-toxic and are acceptable for human pharmaceutical use. Reference to an excipient includes both one and more than one such excipient.

The term “composition” or “pharmaceutical composition” as used herein synonymously includes oral liquid dosage forms such as solutions (aqueous and non-aqueous), suspensions, emulsions, syrups, elixirs, powder for solution, powder for suspension and the like meant for oral administration.

The term “formulation” or “pharmaceutical formulation” as used herein synonymously includes oral liquid dosage forms such as solutions (aqueous and non-aqueous), suspensions, syrups, elixirs, powder for solution, powder for suspension, pellets for oral solution, pellets for oral suspension and the like meant for oral administration.

Oral liquids include, but are not limited to, solutions (both aqueous and nonaqueous), suspensions, syrups, slurries, juices, elixirs, dispersions, powder for solution, powder for suspension, pellets for oral solution, pellets for oral suspension and the like. It is envisioned that solution/suspensions are also included where certain components described herein are in a solution while other components are in a suspension. In some embodiments, the oral liquid formulation is a powder or pellets mixed with a vehicle, before administration.

One aspect relates to an oral liquid pharmaceutical formulation comprising a powder for oral solution or pellets for oral solution prepared with a pharmaceutically acceptable liquid carrier. Another aspect relates to an oral liquid pharmaceutical formulation comprising the powder for oral suspension or pellets for oral suspension prepared with a pharmaceutically acceptable liquid carrier.

Angiotensin converting enzyme (ACE) inhibitors may include Alacepril, Benazepril, Captopril, Ceronapril, Cilazapril, Delapril, Enalapril, Fosinopril, Imidapril, Lisinopril, Moexipril, Moveltipril, Perindopril, Quinapril, Ramipril, Spirapril, Trandolapril, Temocapril, Utibapril, Zofenopril and the like, including their pharmaceutically acceptable salts or their prodrugs or their active moiety or their salt hydrates or other breakdown products well-known to those of skill in the art.

Specific angiotensin converting enzyme inhibitors' salt form may include Benazepril hydrochloride, Enalapril maleate, Quinapril hydrochloride, Moexipril hydrochloride, Lisinopril hydrochloride and Ramipril hydrochloride.

Typical breakdown products of angiotensin converting enzyme inhibitors include, but are not limited to, benazeprilat for benazepril drugs, enalaprilat and/or enalapril-diketopiperazine for enalapril drugs, quinaprilat and/or quinapril-diketopiperazine for quinapril drugs and perindoprilat for perindopril drugs.

In embodiments, ACE inhibitors for incorporation in the novel formulations of the present disclosure are lisinopril hydrochloride and benazepril hydrochloride.

Lisinopril, an angiotensin-converting enzyme (ACE) inhibitor, a synthetic peptide derivative, has IUPAC name; N2-[(1S)-1-carboxy-3-phenylpropyl]-L-lysyl-L-proline and its structural formula is as follows:

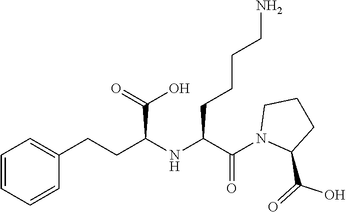 [SMILES: NCCCC[C@H](N[C@@H](CCc1ccccc1)C(=O)O)C(=O)N1CCC[C@H]1C(=O)O]

Lisinopril exhibits polymorphism and it has amorphous and crystalline hydrate forms such as lisinopril amorphous form, lisinopril monohydrate (form-I), lisinopril monohydrate (form-II), and lisinopril dihydrate. In embodiments, Lisinopril is a white to off-white, crystalline powder, with a molecular weight of 441.52 (for dihydrate). It is soluble in water, sparingly soluble in methanol, and practically insoluble in ethanol.

Benazepril hydrochloride is a white to off-white crystalline powder, soluble (greater than 100 mg/mL) in water, in ethanol, and in methanol. Benazepril hydrochloride's chemical name is 3-[[1-(ethoxy carbonyl)-3-phenyl-(1S)-propyl]amino]-2,3,4,5-tetrahydro-2-oxo-1H-1-(3S)-benzazepine-1-acetic acid monohydrochloride; its structural formula is:

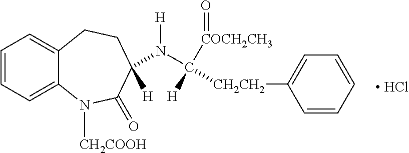

Its empirical formula is C₂₄H₂₈N₂O₅·HCl, and its molecular weight is 460.96. Benazeprilat, the active metabolite of benazepril, is a nonsulfhydryl angiotensin converting enzyme (ACE) inhibitor. Benazepril is converted to benazeprilat by hepatic cleavage of the ester group.

Benazepril is a BCS I compound and is soluble in water, methanol, ethanol, acetic anhydride, glacial acetic acid and the like. It is sensitive to water, and the particle size of the raw material medicine is an important factor influencing the dissolution of the preparation.

Diuretics useful in the novel formulations and methods of treatment of this disclosure are Altizide, Azosemide, Bemetizide, Bendroflumethiazide, Benzthiazide, Butizide, Chlorothiazide, Cicletanide, Clopamide, Cyclopenthiazide, Cyclothiazide, Ethiazide, Furosemide, Hydrochlorothiazide, Hydroflumethiazide, Indapamide, Methyclothiazide, Penflutizide, Piretanide, Polythiazide, Trichlormethiazide, Triflumethiazide and the like, including their pharmaceutically acceptable salts or their prodrugs or their active moiety or their salt hydrates or other breakdown products well-known to those of skill in the art.

In embodiments, diuretics for incorporation in the novel formulations of the present disclosure is hydrochlorothiazide.

Hydrochlorothiazide is a thiazide derivative diuretic which is used in the treatment of edema and hypertension. Thiazide derivative diuretic increases elimination of sodium, chloro and water by inhibiting the passage of sodium from kidney tubules. The diuretic effect of Hydrochlorothiazide indirectly decreases the plasma volume with the elevations of plasma renin activity, aldosterone secretion and loss of urinary potassium and with the decreases of serum potassium level.

The hydrochlorothiazide of the present disclosureincludes pharmacologically acceptable salts thereof, for example, a hydrohalogenic acid salt such as hydrofluoride, hydrochloride, hydrobromide or hydroiodide; nitrate; perchlorate; sulfate; phosphate; a C1-C4 alkanesulfonic acid salt, which may be optionally substituted with a halogen atom(s) such as methanesulfonate, trifluoromethanesulfonate or ethanesulfonate; a C6-C10 arylsulfonic acid salt, which may be optionally substituted with a C1-C4 alkyl group(s), such as benzenesulfonate or p-toluenesulfonate; a C1-C6 aliphatic acid salt such as acetate, malate, fumarate, succinate, citrate, tartrate, oxalate or maleate; or an amino acid salt such as the glycine salt, lysine salt, alginine salt, ornitine salt, glutamic acid salt or aspartic acid salt.

Hydrochlorothiazide, IUPAC name is 6-chloro-3,4-dihydro-2H-1,2,4-benzothiadiazine-7-sulfonamide-1,1-dioxide, having structural formula is as follows:

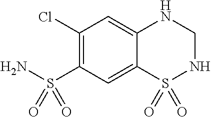 [SMILES: NS(=O)(=O)c1cc2c(cc1Cl)NCNS2(=O)=O]

Its empirical formula is C₇H₈ClN₃O₄S₂; molecular weight is 297.74 and its white, or practically white, crystalline powder, which is slightly soluble in water, but freely soluble in sodium hydroxide solution.

Hydrochlorothiazide is a BSC IV compound, but can be considered to have high solubility, based on 25 mg and solubility studies as the highest dose and low permeability class III compounds.

One embodiment of the present disclosure relates to stable oral liquid pharmaceutical formulations comprising a therapeutically effective amount of angiotensin converting enzyme inhibitor and/or diuretic or their pharmaceutically acceptable salts thereof, one or more sweetening agent(s) and at least one pharmaceutically acceptable excipient.

Another embodiment of the present disclosure relates to stable liquid pharmaceutical formulations for oral administration comprising a therapeutically effective amount of angiotensin converting enzyme inhibitor and/or diuretic or their pharmaceutically acceptable salts thereof and a sweetening agent(s) selected from glucose, fructose, sucrose, xylitol, tagatose, sucralose, maltitol, isomaltulose, isomalt, lactitol, sorbitol, erythritol, liquid glucose, compressible sugar, confectioner's sugar, dextrose, maltitol solution, a maltitol oligomer, maltose, mannitol, neohesperidin thaumatin, trehalose, magnasweet, xylitol, trehalose, maltodextrin, polydextrose, glycerin, inulin, maltol, acesulfame and its salts, alitame, aspartame, neotame, sodium cyclamate, saccharin and its salts, stevioside, monkfruit, hydrogenated starch hydrolysates, maltitol syrup, high fructose corn syrup, prosweet, invertose, ora-sweet, ora-sweet-SF, flavored syrups, and the like or their combinations of two or more.

Another embodiment of the present disclosure relates to stable liquid pharmaceutical formulations for oral administration comprising a therapeutically effective amount of angiotensin converting enzyme inhibitor and/or diuretic and a sweetening agent(s) comprising an amount of about 5 to 70% w/w.

Another embodiment of the present disclosure relates to stable oral liquid pharmaceutical formulations comprising a therapeutically effective amount of angiotensin converting enzyme inhibitor and/or diuretic or their pharmaceutically acceptable salts thereof, a stabilizing agent(s) and at least one pharmaceutically acceptable excipient.

A stabilizing agent is a substance used to maintain the physical and chemical properties of a particle, solution or drug, preventing degradation and extending its useful shelf life. Stabilizing agents play a crucial role in enhancing the properties of drugs. Many drugs have poor water solubility, which can limit their effectiveness. Stabilizing agents can form inclusion complexes with these drugs, significantly improving their solubility and, consequently, their bioavailability. They can prevent oxidation or hydrolysis, thereby extending the shelf life of the drug. By forming complexes with metal ions or other potentially harmful substances, stabilizing agents can reduce the toxicity of certain drugs. Stabilizing agents can improve the delivery of drugs to their target sites in the body. This can be particularly useful for drugs that need to cross biological membranes or be delivered to specific tissues.

Another embodiment of the present disclosure relates to stable liquid pharmaceutical formulations for oral administration comprising a therapeutically effective amount of angiotensin converting enzyme inhibitor and/or diuretic or their pharmaceutically acceptable salts thereof and a stabilizing agent(s) selected from poly-L-lysine, ethylenediaminetetraacetic acid, cyclodextrins (unmodified cyclodextrins such as α-cyclodextrin, β-cyclodextrin and γ-cyclodextrin; substituted cyclodextrins such as partly substituted hydroxyalkyl cyclodextrins, such as hydroxypropyl cyclodextrin or dihydroxypropyl cyclodextrin, partly substituted carboxyalkyl cyclodextrins, such as carboxymethyl cyclodextrin, alkali metal salts of partly substituted carboxyalkyl cyclodextrins or partly substituted water-soluble alkyl cyclodextrins, such as C1 to C4 alkyl cyclodextrins; hydroxypropyl-β-cyclodextrins, sulfobutylether-β-cyclodextrins; methylated β-cyclodextrins such as heptakis (2,6-di-O-methyl)-β-cyclodextrin), RAMEB (statistically methylated or randomly-methylated-β-cyclodextrin β-cyclodextrin), TRIMEB (heptakis (2,3,6-tri-O-methyl)-β-cyclodextrin) and CRYSMEB (weakly methylated β-cyclodextrin) and the like), phosphates, zeolites, ammonia, polyvinylpyrrolidone, sodium alginate, gelatin, silicon dioxide and the like or their combinations of two or more.

Another embodiment of the present disclosure relates to stable liquid pharmaceutical formulations for oral administration comprising a therapeutically effective amount of angiotensin converting enzyme inhibitor and/or diuretic and a stabilizing agent(s) comprising an amount of about 0.5 to 30% w/w.

In embodiments, the stable liquid pharmaceutical formulations may include complexing agents, solubilizing agents, stabilizing agents and chelating agents which interact with one another and with the drug components to enhance solubility and stability, thereby forming the stable oral liquid pharmaceutical formulations of the present disclosure.

In embodiments, the stable liquid pharmaceutical formulations may include viscosity modifiers, also known as viscosity thickeners, viscosity enhancers or viscosity-increasing agents, to improve the consistency and stability of liquid formulations. Increasing the viscosity of an oral liquid formulation, by adding a viscosity agent, may limit the diffusion of bitter tasting substances from the saliva to the taste buds.

Another embodiment of the present disclosure relates to stable liquid pharmaceutical formulations for oral administration comprising a therapeutically effective amount of angiotensin converting enzyme inhibitor and/or diuretic or their pharmaceutically acceptable salts thereof and a viscosity modifier(s) selected from hydrocolloid gums, cellulose derivatives, hydroxyethyl cellulose, methylcellulose, carboxymethylcellulose, carbomers, polyvinyl alcohols, silicon dioxide, xanthan gum, crospovidone and the like or their combinations of two or more.

Another embodiment of the present disclosure relates to stable liquid pharmaceutical formulations for oral administration comprising a therapeutically effective amount of angiotensin converting enzyme inhibitor and/or diuretic and a viscosity modifier(s) comprising an amount of about 0.05 to 15% w/w.

Another embodiment of the present disclosure relates to stable oral liquid pharmaceutical formulations comprising a therapeutically effective amount of angiotensin converting enzyme inhibitor and/or diuretic or their pharmaceutically acceptable salts thereof, a flavoring agent(s) and at least one pharmaceutically acceptable excipient.

Another embodiment of the present disclosure relates to stable liquid pharmaceutical formulations for oral administration comprising a therapeutically effective amount of angiotensin converting enzyme inhibitor and/or diuretic or their pharmaceutically acceptable salts thereof and a flavoring agent(s) selected from almond, anise, apple, apricot, bergamot, blackberry, blackcurrant, blueberry, caramel, cherry, cinnamon, clove, coffee, coriander, cranberry, cumin, dill, eucalyptus, fennel, fig, ginger, grape, grapefruit, guava, hop, lemon, licorice, lime, malt, mandarin, molasses, nutmeg, mixed berry, orange, peach, pear, peppermint, pineapple, raspberry, rose, spearmint, strawberry, tangerine, tea, cinnamon oil, oil of wintergreen, peppermint oils, clove oil, bay oil, anise oil, eucalyptus, thyme oil, cedar leaf oil, oil of nutmeg, oil of sage, oil of bitter almonds, and cassia oil and the like or any combinations thereof; vanilla, citrus oil, fruit essences, including apple, banana, pear, peach, strawberry, raspberry, cherry, plums pineapple, apricot, peppermint, tutti frutti, mixed berry, and so forth and the like or any combinations thereof. Solid forms, such as spray dried forms of flavoring agents, may also be useful in the liquid dosage forms disclosed herein.

Another embodiment of the present disclosure relates to stable liquid pharmaceutical formulations for oral administration comprising a therapeutically effective amount of angiotensin converting enzyme inhibitor and/or diuretic and a flavoring agent comprising an amount of about 0.05 to 10% w/w.

Another embodiment of the present disclosure relates to stable oral liquid pharmaceutical formulations comprising a therapeutically effective amount of angiotensin converting enzyme inhibitor and/or diuretic or their pharmaceutically acceptable salts thereof, one or more preservatives and at least one pharmaceutically acceptable excipient. A preservative may be provided to prevent contamination, e.g., growth of microbes, of the formulation.

Another embodiment of the present disclosure relates to stable liquid pharmaceutical formulations for oral administration comprising a therapeutically effective amount of angiotensin converting enzyme inhibitor and/or diuretic or their pharmaceutically acceptable salts thereof and a preservative selected from, erythorbic acid, fumaric acid, malic acid, sorbic acid, propyl gallate, ethyl oleate, methionine, monothioglycerol, thymol, tocopherols, sodium bisulfate, sodium metabisulfite, sodium sulfite, parabens (methylparaben, ethylparaben, propylparaben, butylparaben, isobutylparaben and the like) and/or their salts, benzoic acid, sodium benzoate, potassium sorbate, vanillin, benzyl alcohol, boric acid, calcium acetate, bentonite, cetrimide, chlorhexidine, cetylpyridinium chloride, cresol, chlorbutanol, magnesium trisilicate and the like or their combinations of two or more.

Another embodiment of the present disclosure relates to stable liquid pharmaceutical formulations for oral administration comprising a therapeutically effective amount of angiotensin converting enzyme inhibitor and/or diuretic and a preservative comprising an amount of about 0.01 to 2% w/w.

Another embodiment of the present disclosure relates to stable oral liquid pharmaceutical formulations comprising a therapeutically effective amount of angiotensin converting enzyme inhibitor and/or diuretic or their pharmaceutically acceptable salts thereof, one or more buffering agents and at least one pharmaceutically acceptable excipient. A buffer or buffering agent may be provided in the formulation to maintain a desired pH. In embodiments, the buffer does not affect taste masking.

Another embodiment of the present disclosure relates to stable liquid pharmaceutical formulations for oral administration comprising a therapeutically effective amount of angiotensin converting enzyme inhibitor and/or diuretic or their pharmaceutically acceptable salts thereof and a buffering agent(s) selected from sodium bicarbonate, potassium bicarbonate, magnesium hydroxide, magnesium lactate, magnesium gluconate, aluminum hydroxide, aluminum glycinate, citric acid, fumaric acid, tartaric acid, maleic acid, lactic acid, hydrochloric acid, phosphoric acid, glycine, glacial acetic acid, sodium acetate trihydrate, trisodium citrate, potassium chloride, hydroxymethyl aminomethane, sodium hydroxide, sodium citrate, sodium tartrate, sodium acetate, sodium carbonate, sodium phosphate monobasic, potassium phosphate, sodium polyphosphate, potassium polyphosphate, sodium pyrophosphate, potassium pyrophosphate, disodium hydrogen phosphate, dipotassium hydrogen phosphate, trisodium phosphate, tri potassium phosphate, potassium metaphosphate, magnesium oxide, magnesium carbonate, magnesium silicate, calcium acetate, calcium glycerophosphate, calcium chloride, calcium hydroxide, calcium lactate, calcium carbonate, calcium bicarbonate, and the like or their combinations of two or more.

Another embodiment of the present disclosure relates to stable liquid pharmaceutical formulations for oral administration comprising a therapeutically effective amount of angiotensin converting enzyme inhibitor and/or diuretic and a buffering agent comprising an amount of about 0.05 to 10% w/w.

Another embodiment of the present disclosure relates to stable oral liquid pharmaceutical formulations comprising a therapeutically effective amount of angiotensin converting enzyme inhibitor and/or diuretic or their pharmaceutically acceptable salts thereof, a wetting agent(s) and at least one pharmaceutically acceptable excipient.

Wetting agents are crucial in pharmaceutical formulations to ensure that solid particles remain uniformly distributed within a liquid medium, maintaining the physical stability of the product. These agents are selected based on their ability to maintain the stability of the suspension, prevent sedimentation, and ensure the uniform distribution of the active pharmaceutical ingredient(s).

Suitable wetting agent/suspension aids/suspension stabilizer/gelling agent/thickeners include, but are not limited to, poloxamer, cremophor, methylcellulose, microcrystalline cellulose, sodium carboxymethylcellulose, acacia, xanthan gum, bentonite, dextrin, cellulose derivatives (carboxymethylcellulose and its salts, ethylcellulose, hydroxypropyl methylcellulose, hydroxyethyl cellulose, methylcellulose, and the like), starches (corn starch, potato starch, maize starch, modified starches, and the like), pectin, polyethylene glycol, polyethylene oxide, trehalose, certain silicates (magnesium aluminum silicate, aluminum silicate, etc, such as veegum, bentonite, and kaolin) and certain gums (xanthan gum, guar gum, gum arabic, locust bean gum, etc.), silicon dioxide, microcrystalline cellulose, polyvinylpyrrolidone, 3-butoxy-2-hydroxypropylhydroxyethylcellulose, acrylamide homo- and copolymers, acrylic acid homo- and copolymer, alginates, carboxymethyl hydroxyethyl cellulose, carboxy-vinyl copolymers, cellulose, microcrystalline cellulose (of different grades), hydrophobically modified hydroxyethyl cellulose, hydroxypropyl guar, hydroxypropyl methylcellulose, hydroxypropyl cellulose, partially and fully hydrolyzed polyvinyl alcohols, partially neutralized polyacrylic acid, polyalkylene glycol, polysaccharide gums, polyvinylpyrrolidones, starches, vinylpyrrolidone homo- and copolymers, crospovidone, carbomer water-soluble cellulose ethers, bentonite, laponites, kaolinite, dickite, nacrite, pyrophylite, talc, vermiculite, sauconite, saponite, nontronite, montmorillonite, organically modified montmorillonite clays and the like or their combinations of two or more in an amount of about 0.05 to 15% w/w.

In embodiments, a solvent suitable for the formulations of the present disclosure include, but are not limited to, purified water, alcohol, isopropyl alcohol and the like or their combinations of two or more in an amount to produce 100% for oral administration.

Examples of a co-solvent include, but are not limited to, glycerin, an alcohol (short-chain substituted or non-substituted alcohols such as ethanol, isopropanol, or propanol and the like), a glycol (propylene glycol, polyethylene glycol and the like), and the like or their combinations of two or more in an amount to produce 100% for oral administration.

In embodiments, inert pellets may be included in the formulations of the present disclosure, and may include, but are not limited to, mannitol, sugar, microcrystalline cellulose, vegetable gums, silicas, waxes and the like or their combinations of two or more in pharmaceutically acceptable quantity.

In embodiments, coating agents may be included in the formulations of the present disclosure and may include their different grades, but are not limited to, opadry, poly(vinyl alcohol), poly(vinyl pyrrolidone), hydroxy methyl cellulose, hydroxyethyl cellulose, hydroxypropyl methyl cellulose, hydroxy propyl cellulose, ethylcellulose, methyl cellulose, carboxymethyl cellulose, plasdone, eudragits, phthalates, cellulose acetate phthalate, hydroxypropyl methylcellulose phthalate, hydroxypropyl methylcellulose acetate, hydroxypropyl methylcellulose succinate, methacrylic and/or acrylic polymers, methacrylic acid copolymers, amino methacrylate copolymers, polydextrose, gum acacia and the like or their combinations of two or more in a pharmaceutical acceptable quantity.

Examples of additional excipients which may be included in the formulations of the present disclosure include lubricant (talc, stearates and the like); anticaking agents (crospovidone, colloidal silicon dioxide and the like); resin and combinations thereof in a pharmaceutical acceptable quantity.

Additional excipients are contemplated in the stable oral liquid pharmaceutical formulation embodiments. These additional excipients are selected based on function and compatibility with the oral liquid formulations described herein and may be found, for example in Remington: The Science and Practice of Pharmacy, Nineteenth Ed (Easton, Pa.: Mack Publishing Company, 1995); Hoover, John E., Remington's Pharmaceutical Sciences, (Easton, Pa.: Mack Publishing Co 1975); Liberman, H. A. and Lachman, L., Eds., Pharmaceutical Dosage Forms (New York, N.Y.: Marcel Decker 1980); and Pharmaceutical Dosage Forms and Drug Delivery Systems, Seventh Ed (Lippincott Williams & Wilkins 1999), herein incorporated by reference in their entirety.

One aspect relates to stable oral liquid pharmaceutical formulations comprising angiotensin converting enzyme inhibitor and/or diuretic with one or more pharmaceutically acceptable excipients for the treatment of cardiovascular and other related diseases including hypertension, coronary artery disease, heart failure and acute myocardial infarction.

In aspects stable oral liquid pharmaceutical formulations may comprise:
(i) Lisinopril/Benazepril/Hydrochlorothiazide, a pharmaceutically acceptable salt thereof, a hydrate thereof, or a solvate thereof; and
(iii) a sweetening agent and/or stabilizing agent and/or viscosity modifier.

In aspects, stable oral liquid pharmaceutical formulations may comprise:
(i) Lisinopril/Benazepril, a pharmaceutically acceptable salt thereof, a hydrate thereof, or a solvate thereof;
(ii) Hydrochlorothiazide, a pharmaceutically acceptable salt thereof, a hydrate thereof, or a solvate thereof; and
(iii) a sweetening agent.

In aspects, stable oral liquid pharmaceutical formulations may comprise:
(i) Lisinopril/Benazepril, a pharmaceutically acceptable salt thereof, a hydrate thereof, or a solvate thereof;
(ii) Hydrochlorothiazide, a pharmaceutically acceptable salt thereof, a hydrate thereof, or a solvate thereof; and
(iii) a stabilizing agent.

In aspects, stable oral liquid pharmaceutical formulations may comprise:
(i) Lisinopril/Benazepril, a pharmaceutically acceptable salt thereof, a hydrate thereof, or a solvate thereof;
(ii) Hydrochlorothiazide, a pharmaceutically acceptable salt thereof, a hydrate thereof, or a solvate thereof; and
(iii) a viscosity modifier.

In aspects, a stable oral liquid pharmaceutical formulations may comprise:
(i) Lisinopril/Benazepril, a pharmaceutically acceptable salt thereof, a hydrate thereof, or a solvate thereof;
(ii) Hydrochlorothiazide, a pharmaceutically acceptable salt thereof, a hydrate thereof, or a solvate thereof;
(iii) a sweetening agent;
(iv) a stabilizing agent; and
(v) a viscosity modifier.

In aspects, stable oral liquid pharmaceutical formulation may comprise:
(i) Lisinopril/Benazepril/Hydrochlorothiazide, a pharmaceutically acceptable salt thereof, a hydrate thereof, or a solvate thereof;
(iii) a sweetening agent;
(iv) a stabilizing agent;
(v) a viscosity modifier; and
(vi) one or more pharmaceutical acceptable excipients which include but is not limited to flavoring agent, preservative, buffering agent, wetting agent, solvent, cosolvent, chelating agent, inert pellet, coating agent, lubricant, anticaking agent and the like.

In aspects, stable oral liquid pharmaceutical formulations comprise:
(i) Lisinopril/Benazepril, a pharmaceutically acceptable salt thereof, a hydrate thereof, or a solvate thereof;
(ii) Hydrochlorothiazide, a pharmaceutically acceptable salt thereof, a hydrate thereof, or a solvate thereof;
(iii) a sweetening agent;
(iv) a stabilizing agent;
(v) a viscosity modifier; and
(vi) one or more pharmaceutical acceptable excipients which include but is not limited to flavoring agent, preservative, buffering agent, wetting agent, solvent, cosolvent, chelating agent, inert pellet, coating agent, lubricant, anticaking agent and the like.

In embodiments, the stable oral liquid pharmaceutical formulations comprise:
(i) Lisinopril (about 0.1% to about 2% w/w)/Benazepril (about 0.1% to about 2% w/w)/Hydrochlorothiazide (about 0.15% to about 2.6% w/w), a pharmaceutically acceptable salt thereof, a hydrate thereof, or a solvate thereof;
(ii) about 0.5% to about 70% w/w of a sweetening agent; and/or
(iii) about 0.5% to about 30% w/w of a stabilizing agent; and/or
(iv) about 0.05% to about 15% w/w of a viscosity modifier.

In embodiments, the stable oral liquid pharmaceutical formulations comprise:
(i) about 0.1% to about 2% w/w of Lisinopril/Benazepril, a pharmaceutically acceptable salt thereof, a hydrate thereof, or a solvate thereof;
(ii) about 0.15% to about 2.6% w/w of Hydrochlorothiazide, a pharmaceutically acceptable salt thereof, a hydrate thereof, or a solvate thereof, and
(iii) about 0.5% to about 70% w/w of a sweetening agent.

In embodiments, the stable oral liquid pharmaceutical formulations comprise:
(i) about 0.1% to about 2% w/w of Lisinopril/Benazepril, a pharmaceutically acceptable salt thereof, a hydrate thereof, or a solvate thereof;
(ii) about 0.15% to about 2.6% w/w of Hydrochlorothiazide, a pharmaceutically acceptable salt thereof, a hydrate thereof, or a solvate thereof; and
(iii) about 0.5% to about 30% w/w of a stabilizing agent.

In embodiments, the stable oral liquid pharmaceutical formulations comprise:
(i) about 0.1% to about 2% w/w of Lisinopril/Benazepril, a pharmaceutically acceptable salt thereof, a hydrate thereof, or a solvate thereof;
(ii) about 0.15% to about 2.6% w/w of Hydrochlorothiazide, a pharmaceutically acceptable salt thereof, a hydrate thereof, or a solvate thereof; and
(iii) about 0.05% to about 15% w/w of a viscosity modifier.

In embodiments, the stable oral liquid pharmaceutical formulations comprise:
(i) about 0.1% to about 2% w/w of Lisinopril/Benazepril, a pharmaceutically acceptable salt thereof, a hydrate thereof, or a solvate thereof;
(ii) about 0.15% to about 2.6% w/w of Hydrochlorothiazide, a pharmaceutically acceptable salt thereof, a hydrate thereof, or a solvate thereof;
(iii) about 0.5% to about 70% w/w of a sweetening agent; and
(iv) about 0.5% to about 30% w/w of a stabilizing agent.

In embodiments, the stable oral liquid pharmaceutical formulations comprise:
(i) about 0.1% to about 2% w/w of Lisinopril/Benazepril, a pharmaceutically acceptable salt thereof, a hydrate thereof, or a solvate thereof;
(ii) about 0.15% to about 2.6% w/w of Hydrochlorothiazide, a pharmaceutically acceptable salt thereof, a hydrate thereof, or a solvate thereof;
(iii) about 0.5% to about 70% w/w of a sweetening agent;
(iv) about 0.5% to about 30% w/w of a stabilizing agent; and
(v) about 0.05% to about 15% w/w of a viscosity modifier.

In embodiments, the stable oral liquid pharmaceutical formulations comprise:
(i) Lisinopril (about 0.1% to about 2% w/w)/Benazepril (about 0.1% to about 2% w/w)/Hydrochlorothiazide (about 0.15% to about 2.6% w/w), a pharmaceutically acceptable salt thereof, a hydrate thereof, or a solvate thereof;
(ii) about 0.5% to about 70% w/w of a sweetening agent;
(iii) about 0.5% to about 30% w/w of a stabilizing agent;
(iv) about 0.05% to about 15% w/w of a viscosity modifier; and
(v) one or more pharmaceutical acceptable excipients which include, but is not limited to a flavoring agent (about 0.05-10% w/w), preservative (about 0.01-2% w/w), a buffering agent (about 0.05-10% w/w), a wetting agent (about 0.05-15% w/w), a solvent (q.s.), a cosolvent (q.s.), an inert pellet (about 1-15% w/w), a coating agent (about 0.5-30% w/w), a lubricant (about 0.01-5% w/w), an anticaking agent (about 0.05-10% w/w) and the like and their combinations thereof.

In embodiments, the stable oral liquid pharmaceutical formulations comprise:
(i) about 0.1% to about 2% w/w of Lisinopril/Benazepril, a pharmaceutically acceptable salt thereof, a hydrate thereof, or a solvate thereof;
(ii) about 0.15% to about 2.6% w/w of Hydrochlorothiazide, a pharmaceutically acceptable salt thereof, a hydrate thereof, or a solvate thereof;
(iii) about 0.5% to about 70% w/w of a sweetening agent;
(iv) about 0.5% to about 30% w/w of a stabilizing agent;
(v) about 0.05% to about 15% w/w of a viscosity modifier; and
(vi) one or more pharmaceutical acceptable excipients which include but is not limited to a flavoring agent (about 0.05-10% w/w), a preservative (about 0.01-2% w/w), a buffering agent (about 0.05-10% w/w), a wetting agent (about 0.05-15% w/w), a solvent (q.s.), a cosolvent (q.s.), an inert pellet (about 1-15% w/w), a coating agent (about 0.5-30% w/w), a lubricant (about 0.01-5% w/w), an anticaking agent (about 0.05-10% w/w) and the like and their combinations thereof.

Also provided herein are methods of treating hypertension comprising administering to a patient in need thereof a lisinopril/benazepril and/or hydrochlorothiazide stable oral liquid pharmaceutical formulation described herein. In some embodiments, the hypertension is primary (essential) hypertension. In some embodiments, the hypertension is secondary hypertension. In some embodiments, the subject with hypertension has blood pressure values greater than or equal to 140/90 mmm Hg.

Another aspect disclosed herein relates to a method for the symptomatic treatment of a chronic stable angina in a patient in need thereof, which comprises administering to said patient a therapeutically effective amount of any one of the formulations disclosed herein.

Yet another aspect disclosed herein relates to a method for the treatment of confirmed or suspected vasospastic angina in a patient in need thereof, which comprises administering to said patient a therapeutically effective amount of any one of the formulations disclosed herein.

Yet another aspect disclosed herein relates to a method of treating heart failure or acute myocardial infarction comprising administering to a patient in need thereof a lisinopril/benazepril and/or hydrochlorothiazide oral liquid formulation described herein.

### EXAMPLES

The following examples further describe and demonstrate particular embodiments within the scope of the present invention. The examples are given solely for illustration and are not to be construed as limitations as many variations are possible without departing from the spirit and scope of the invention.

### A. General Formulations of the Invention

| Formulation 1 | % w/w | Formulation 2 | % w/w |
|---|---|---|---|
| Lisinopril/Benazepril | 0.1-2 | Lisinopril/Benazepril | 0.1-2 |
| Sweetening agent | 0.5-70 | Stabilizing agent | 0.5-30 |
| Solvent | Q.S. | Solvent | Q.S. |
| and/or cosolvent | | and/or cosolvent | |

| Formulation 3 | % w/w | Formulation 4 | % w/w |
|---|---|---|---|
| Hydrochlorothiazide | 0.15-2.6 | Hydrochlorothiazide | 0.15-2.6 |
| Sweetening agent | 0.5-70 | Stabilizing agent | 0.5-30 |
| Solvent | Q.S. | Solvent | Q.S. |
| and/or cosolvent | | and/or cosolvent | |

| Formulation 5 | % w/w | Formulation 6 | % w/w |
|---|---|---|---|
| Lisinopril/Benazepril | 0.1-2 | Lisinopril/Benazepril | 0.1-2 |
| Hydrochlorothiazide | 0.15-2.6 | Hydrochlorothiazide | 0.15-2.6 |
| Sweetening agent | 0.5-70 | Stabilizing agent | 0.5-30 |
| Solvent | Q.S. | Solvent | Q.S. |
| and/or cosolvent | | and/or cosolvent | |

| Formulation 7 | % w/w |
|---|---|
| Lisinopril/Benazepril | 0.1-2 |
| Hydrochlorothiazide | 0.15-2.6 |
| Viscosity modifiers | 0.05-15 |
| Solvent | Q.S. |
| and/or cosolvent | |

| Formulation 8 | % w/w | Formulation 9 | % w/w |
|---|---|---|---|
| Lisinopril/Benazepril | 0.1-2 | Hydrochlorothiazide | 0.15-2.6 |
| Viscosity modifiers | 0.05-15 | Viscosity modifiers | 0.05-15 |
| Solvent | Q.S. | Solvent | Q.S. |
| and/or cosolvent | | and/or cosolvent | |

| Formulation 10 | % w/w | Formulation 11 | % w/w |
|---|---|---|---|
| Lisinopril/Benazepril | 0.1-2 | Lisinopril/Benazepril | 0.1-2 |
| Sweetening agent | 0.5-70 | Sweetening agent | 0.5-70 |
| Stabilizing agent | 0.5-30 | Stabilizing agent | 0.5-30 |
| Solvent | Q.S. | Viscosity modifiers | 0.05-15 |
| and/or cosolvent | | Solvent | Q.S. |
| | | and/or cosolvent | |

| Formulation 12 | % w/w | Formulation 13 | % w/w |
|---|---|---|---|
| Hydrochlorothiazide | 0.15-2.6 | Hydrochlorothiazide | 0.15-2.6 |
| Sweetening agent | 0.5-70 | Sweetening agent | 0.5-70 |
| Stabilizing agent | 0.5-30 | Stabilizing agent | 0.5-30 |
| Solvent | Q.S. | Viscosity modifiers | 0.05-15 |
| and/or cosolvent | | Solvent | Q.S. |
| | | and/or cosolvent | |

| Formulation 14 | % w/w | Formulation 15 | % w/w |
|---|---|---|---|
| Lisinopril/Benazepril | 0.1-2 | Lisinopril/Benazepril | 0.1-2 |
| Hydrochlorothiazide | 0.15-2.6 | Hydrochlorothiazide | 0.15-2.6 |
| Sweetening agent | 0.5-70 | Sweetening agent | 0.5-70 |
| Stabilizing agent | 0.5-30 | Stabilizing agent | 0.5-30 |
| Solvent and/or | Q.S. | Viscosity modifiers | 0.05-15 |
| cosolvent | | Solvent and/or | Q.S. |
| | | cosolvent | |

### B. Specific Formulation of the Invention

| Ingredients | % w/w | Ingredients | % w/w |
|---|---|---|---|
| Lisinopril/Benazepril | 0.1-2 | Hydrochlorothiazide | 0.15-2.6 |
| Sweetening agent | 0.5-70 | Sweetening agent | 0.5-70 |
| Stabilizing agent | 0.5-30 | Stabilizing agent | 0.5-30 |
| Viscosity modifiers | 0.05-15 | Viscosity modifiers | 0.05-15 |
| Excipient* | Q.S. | Excipient* | Q.S. |

| Ingredients | % w/w |
|---|---|
| Lisinopril/Benazepril | 0.1-2 |
| Hydrochlorothiazide | 0.15-2.6 |
| Sweetening agent | 0.5-70 |
| Stabilizing agent | 0.5-30 |
| Viscosity modifiers | 0.05-15 |
| Excipient* | Q.S. |

* one or more pharmaceutical acceptable excipients which include but is not limited to a flavoring agent (about 0.05-10% w/w), a preservative (about 0.01-2% w/w), a buffering agent (about 0.05-10% w/w), a wetting agent (about 0.05-15% w/w), a solvent (q.s.), a cosolvent (q.s.), an inert pellet (about 1-15% w/w), a coating agent (about 0.5-30% w/w), a lubricant (about 0.01-5% w/w), an anticaking agent (about 0.05-10% w/w) and the like and their combinations thereof.

### Example 1: Lisinopril+HCTZ Oral Solution

| Ingredients | Function | % W/W |
|---|---|---|
| Lisinopril | Active pharmaceutical | 0.1-2 |
| | ingredient | |
| Hydrochlorothiazide | Active pharmaceutical | 0.15-2.6 |
| | ingredient | |
| Sodium benzoate | Preservative | 0.05-2 |
| Xylitol/Mannitol/ | Sweetening agent | 0.5-50 |
| Sorbitol/Sucralose | | |
| Glycerol | Cosolvent | 10-80 |
| Citric acid | Buffering agent | 0.05-5 |
| Sodium citrate | Buffering agent | 0.1-5 |
| Cremophor/ | Wetting agent | 0.1-10 |
| Poloxamer/PEG | | |
| Hydroxy | Stabilizing agent | 0.5-30 |
| β-cyclodextrin/Povidone | | |
| Propylene glycol | Cosolvent | 5-30 |
| Poly-L-lysine | Stabilizing agent | 0.005-0.1 |
| Hydroxyethyl | Viscosity modifier | 0.05-7 |
| cellulose/Xanthan gum | | |
| Strong peppermint/ | Flavoring agent | 0.05-3 |
| Polomint flavor | | |
| Orange flavor | Flavoring agent | 0.05-3 |
| Citrus flavor | Flavoring agent | 0.05-3 |
| HCl//NaoH | pH modifier | q.s. |
| Purified water | Solvent | q.s. |

### General Manufacturing Process for Oral Solution

An oral liquid pharmaceutical formation was prepared according to the following procedure:
1. Preparation of Cyclodextrin Solution: Hydroxy β-cyclodextrin was added to purified water in a separate vessel and mixed until completely dissolved to obtain a clear solution.
2. Addition of Hydrochlorothiazide: hydrochlorothiazide was added to the solution of step 1 and mixed until completely dissolved to obtain a clear solution.
3. Addition of preservative and co-solvent: Sodium benzoate or poly-l lysine and propylene glycol were added to the step 2 solution and mixed until fully dissolved to provide a clear solution.
4. Preparation of Polymer Solution: Hydroxyethyl cellulose or xanthan gum was added to glycerol in a separate vessel and mixed until completely dissolved to obtain a clear polymer solution.
5. Addition of Lisinopril: Lisinopril was added to the step 4 solution and mixed until completely dissolved to form a clear solution.
6. Addition of Sweeteners: Xylitol or mannitol or sorbitol was added to the step 5 solution under continuous stirring until completely mixed to form a clear solution.
7. Addition of Buffering Agents and Flavor: Citric acid, sodium citrate and flavoring agents were added to the step 6 solution under continuous stirring and mixed to form a clear solution.
8. Volume adjustment: The solution of step 7 was brought to final volume using purified water.
9. Filling: The resulting clear solution was filled into bottles.

### Example 2: Lisinopril+HCTZ Powder/Pellet for Oral Solution

| Ingredients | Function | % W/W |
|---|---|---|
| Lisinopril | Active pharmaceutical | 0.1-2 |
| | ingredient | |
| Hydrochlorothiazide | Active pharmaceutical | 0.15-2.6 |
| | ingredient | |
| Sodium benzoate | Preservative | 0.05-2 |
| Xylitol/Mannitol/ | Sweetening agent | 0.5-70 |
| Sorbitol/Sucralose | | |
| Citric acid | Buffering agent | 0.05-5 |
| Sodium citrate | Buffering agent | 0.1-5 |
| Cremophor/Poloxamer/PEG | Wetting agent | 0.1-15 |
| Hydroxy | Stabilizing agent | 0.5-30 |
| β-cyclodextrin/Povidone | | |
| Hydroxyethyl | Viscosity modifier | 0.05-7 |
| cellulose/Xanthan gum | | |
| Strong peppermint/ | Flavoring agent | 0.05-3 |
| Polomint flavor | | |
| Orange flavor | Flavoring agent | 0.05-3 |
| Citrus flavor | Flavoring agent | 0.05-3 |
| Purified water | Solvent | q.s. |
| Hydroxyethyl cellulose | Viscosity modifier | 0.1-5 |
| HPMC | Viscosity modifier | 1-15 |
| Hydroxypropyl | Coating agent | 0.5-15 |
| cellulose/HPMC/ | | |
| methacrylic copolymers | | |
| Opadry clear | Coating agent | 0.5-15 |
| (HPMC based) | | |
| Sugar/Mannitol spheres | Inert pellets | 1-15 |
| Isopropyl alcohol | Solvent | q.s. |

### General Manufacturing Process of Powder/Pellet for Oral Solution

### A. Preparation of the Lisinopril Part:

1. Sifting of Dry Materials: Lisinopril dihydrate, maltitol or mannitol, and sodium benzoate were sifted and transferred to a rapid mixer granulator (RMG).
2. Preparation of Binder Solution: The required quantity of an isopropyl alcohol/purified water mixture was dispensed into a stainless steel (SS) vessel. Xanthan Gum was dispersed therein under stirring until a lump free dispersion was obtained.
3. Initial Mixing: The sifted materials of step 1 were mixed in the RMG for 10 minutes with the impeller ON and the chopper OFF.
4. Addition of Binder Solution: The prepared binder solution of step 2 was added to the mixture of step 3 with the impeller ON and the chopper OFF.
5. Kneading: The resulting wet mass was kneaded with both the impeller and chopper ON to form granules.
6. Adjustment of Moisture (if required): Additional isopropyl alcohol/purified water mixture was added as needed to the wet mass obtained in step 5 with the impeller ON and the chopper OFF, followed by kneading with both the impeller and the chopper ON.
7. Drying: The wet mass from step 6 was transferred to a fluid bed dryer (FBD) and dried at a low temperature until the desired loss on drying (LOD) of not more than 2.0% w/w was achieved.
8. Milling: The dried granules of step 7 were milled/sifted using a milling and sifting machine, and the milled granules were collected.

### B. Preparation of Hydrochlorothiazide Part—by Top Spray Technique:

1. Sifting of Dry Materials: Hydrochlorothiazide, maltitol or mannitol, and Hydroxy β-cyclodextrin or povidone were sifted.
2. Preparation of Coating Solution: The required quantity of isopropyl alcohol/water mixture was transferred into a SS vessel. Opadry clear (HPMC) or methacrylic copolymers and Cremophor, poloxamer, or PEG were slowly dispersed under stirring to obtain a uniform solution.
3. Prewarming: The sifted materials from step 1 were loaded into a fluid bed processer and prewarmed at a product temperature of 30° C. for 10±5 mins.
4. Top Spray Coating: The step 3 materials were coated with the solution of step 2 using atop spray technique until the desired weight gain was achieved.
5. Sifting: The coated granules were sifted and collected separately.
6. Sifting of Additional Excipients: Hydroxypropyl cellulose, citric acid monohydrate, sodium citrate and flavoring agents were sifted and collected separately.
7. Blending: The sifted granules of step 5 were transferred into a blender, and the sifted ingredients of step 6 were added, and the mixture was blended.

### C. Preparation of the Hydrochlorothiazide Part—by Wurster (Bottom Spray) Technique:

1. Sifting of Starter Cores: Sugar spheres or mannitol spheres were sifted.
2. Preparation of Coating Solution: The required quantity of isopropyl alcohol/water mixture was transferred into a SS vessel. Opadry clear (HPMC) or methacrylic copolymers and Cremophor Poloxamer or PEG were slowly dispersed under stirring to obtain a uniform solution.
3. Addition of Drug: Hydrochlorothiazide was slowly added to the solution of step 2 under stirring to obtain a uniform drug-containing coating solution.
4. Prewarming: The sifted materials from step 1 were loaded into a fluid bed processer and prewarmed at a product temperature of 30° C. for 10±5 mins.
5. Bottom Spray Coating: The step 4 materials were coated with the drug-containing coating solution using a bottom spray (Wurster) technique until the desired weight gain was achieved.
6. Sifting: The above coated granules were sifted and collected separately.
7. Sifting of Additional Excipients: Hydroxypropyl cellulose, citric acid monohydrate, sodium citrate and flavoring agents were sifted and collected separately.
8. Blending: The sifted granules of step 6 were transferred into blender and sifted ingredients of step 7 were added, and the mixture was blended.
9. Filling: The Lisinopril part granules and the Hydrochlorothiazide part granules were combined and filled into sachets.

### Example 3: Lisinopril+HCTZ Oral Suspension

| Ingredients | Function | % W/W |
|---|---|---|
| Lisinopril | Active pharmaceutical | 0.1-2 |
| | ingredient | |
| Hydrochlorothiazide | Active pharmaceutical | 0.15-2.6 |
| | ingredient | |
| Sodium benzoate | Preservative | 0.05-2 |
| Xylitol/Mannitol/ | Sweetening agent | 0.5-50 |
| Sorbitol/Sucralose | | |
| Glycerol | Cosolvent | 10-80 |
| Citric acid | Buffering agent | 0.05-5 |
| Sodium citrate | Buffering agent | 0.1-5 |
| Cremophor/Poloxamer/PEG | Wetting agent | 0.1-10 |
| Hydroxy | Stabilizing agent | 0.5-30 |
| β-cyclodextrin/Povidone | | |
| Propylene glycol | Cosolvent | 5-30 |
| Poly-L-lysine | Stabilizing agent | 0.005-0.1 |
| Hydroxyethyl | Viscosity modifier | 0.05-7 |
| cellulose/Xanthan gum | | |
| Strong peppermint/ | Flavoring agent | 0.05-3 |
| Polomint flavor | | |
| Orange flavor | Flavoring agent | 0.05-3 |
| Citrus flavor | Flavoring agent | 0.05-3 |
| HCl/NaoH | pH modifier | q.s. |
| Purified water | Solvent | q.s. |
| Microcrystalline cellulose | Viscosity modifier | 0.2-15 |
| Carboxymethylcellulose | Viscosity modifier | 0.05-10 |
| sodium | | |
| Crospovidone/Colloidal | Anticaking agent | 0.05-10 |
| silicon dioxide | | |

### General Manufacturing Process for Oral Suspension

An oral pharmaceutical suspension was prepared according to the following procedure:
1. Preparation of Cyclodextrin Solution: Hydroxy-β-cyclodextrin was added to purified water or isopropyl alcohol in a separate vessel and mixed until completely dissolved to obtain a clear solution.
2. Addition of Hydrochlorothiazide: Hydrochlorothiazide was added to the solution of Step 1 and mixed until fully dissolved, yielding a clear solution.
3. Addition of Preservative: Sodium benzoate was added to the Step 2 solution and mixed until completely dissolved to form a clear solution.
4. Addition of Co-solvent: Propylene glycol was added to the Step 3 solution and mixed until uniformly incorporated to obtain a clear solution.
5. Preparation of Lisinopril Solution: Lisinopril was added to purified water in a separate vessel and mixed until fully dissolved to obtain a clear drug solution.
6. Preparation of Polymer Solution: Hydroxyethyl cellulose or xanthan gum was added to glycerol in a separate vessel and mixed until fully dissolved to obtain a clear polymer solution.
7. Combination of Polymer and Drug-Carrier Solutions: The polymer solution of Step 6 was added to the solution of Step 4 under stirring and mixed until homogeneous.
8. Addition of Sweeteners and Suspending Agents: Maltitol, mannitol, or sucralose, together with microcrystalline cellulose and sodium carboxymethylcellulose, were added to the Step 7 mixture under stirring and mixed until a uniform, lump-free suspension was obtained.
9. Incorporation of Lisinopril Solution: The Lisinopril solution of Step 5 was added to the Step 8 suspension under stirring and mixed until a homogeneous, lump-free suspension was obtained.
10. Addition of Buffering Agents and Other Excipients: Citric acid, sodium citrate, flavoring agents, and crospovidone or colloidal silicon dioxide were added to the Step 9 suspension under stirring and mixed until completely uniform.
11. Volume Adjustment: Purified water was added to the Step 10 suspension to bring the mixture to the final desired volume.
12. Filling: The resulting oral suspension was filled into bottles.

### Example 4: Lisinopril+HCTZ Powder/Pellet for Oral Suspension

| Ingredients | Function | % W/W |
|---|---|---|
| Lisinopril | Active pharmaceutical | 0.1-2 |
| | ingredient | |
| Hydrochlorothiazide | Active pharmaceutical | 0.15-2.6 |
| | ingredient | |
| Sodium benzoate | Preservative | 0.05-2 |
| Xylitol/Mannitol/ | Sweetening agent | 0.5-70 |
| Sorbitol/Sucralose | | |
| Citric acid | Buffering agent | 0.05-5 |
| Sodium citrate | Buffering agent | 0.1-5 |
| Hydroxyethyl | Viscosity modifier | 0.05-7 |
| cellulose/Xanthan gum | | |
| Strong peppermint/ | Flavoring agent | 0.05-3 |
| Polomint flavor | | |
| Orange flavor | Flavoring agent | 0.05-3 |
| Citrus flavor | Flavoring agent | 0.05-3 |
| Purified water | Solvent | q.s. |
| Microcrystalline cellulose | Viscosity modifier | 0.2-15 |
| Carboxymethylcellulose | Viscosity modifier | 0.05-10 |
| sodium | | |
| Crospovidone/Colloidal | Anticaking agent | 0.05-10 |
| silicon dioxide | | |
| Hydroxyethyl cellulose | Viscosity modifier | 0.1-5 |
| HPMC | Viscosity modifier | 1-15 |
| Hydroxypropyl | Coating agent | 0.5-15 |
| cellulose/HPMC/ | | |
| methacrylic copolymers | | |
| Opadry clear (HPMC based) | Coating agent | 0.5-15 |
| Sugar/Mannitol spheres | Inert pellets | 1-15 |
| Isopropyl alcohol | Solvent | q.s. |
| Talc | Lubricant | 0.05-5 |

### General Manufacturing Process of Powder/Pellet for Oral Suspension

### A. Preparation of the Lisinopril Part:

1. Sifting of Dry Materials: Lisinopril dihydrate, maltitol or mannitol, and sodium benzoate were sifted and transferred into a rapid mixer granulator (RMG).
2. Preparation of Binder Solution: The required quantity of an isopropyl alcohol/purified water mixture was dispensed into a stainless-steel vessel. Xanthan gum was dispersed therein under stirring until a lump-free dispersion was obtained.
3. Initial Mixing: The sifted materials of Step 1 were mixed in the RMG for 10 minutes with the impeller on and the chopper off.
4. Addition of Binder Solution: The binder solution of Step 2 was added to the mixture of Step 3 with the impeller on and the chopper off.
5. Kneading: The resulting wet mass was kneaded with both the impeller and chopper on to form granules.
6. Optional Moisture Adjustment: If required, additional isopropyl alcohol/purified water mixture was added to the wet mass obtained in Step 5 with the impeller on and the chopper off, followed by further kneading with the impeller and chopper on.
7. Drying: The wet mass of Step 6 was transferred to a fluid bed dryer (FBD) and dried at a low temperature until a loss on drying (LOD) of not more than 2.0% w/w was achieved.
8. Milling: The dried granules from Step 7 were milled and sifted using a milling and sifting machine, and the resulting granules were collected.

### B. Preparation of the Hydrochlorothiazide Part—by Top Spray Technique:

1. Sifting of Dry Materials: Hydrochlorothiazide and maltitol or mannitol were sifted.
2. Preparation of Coating Solution: The required quantity of an isopropyl alcohol/water mixture was transferred to a stainless-steel vessel. Opadry clear (HPMC) or methacrylic copolymers were slowly dispersed under stirring until a uniform solution was obtained.
3. Prewarming: The sifted materials from Step 1 were loaded into a fluid bed processor and prewarmed at a product temperature of 30° C. for 10±5 minutes.
4. Top Spray Coating: The materials from Step 3 were coated with the solution of Step 2 using a top-spray technique until the desired weight gain was achieved.
5. Sifting: The coated granules were sifted and collected separately.
6. Sifting of Additional Excipients: Hydroxypropyl cellulose, citric acid, sodium citrate, microcrystalline cellulose, sodium carboxymethylcellulose, and flavoring agents were sifted and collected separately.
7. Blending: The coated granules of Step 5 were transferred to a blender. The sifted excipients from Step 6 were added, and the mixture was blended.

### C. Preparation of the Hydrochlorothiazide Part-Processing by Wurster Technique:

1. Sifting of Starter Cores: Sugar spheres or mannitol spheres were sifted.
2. Preparation of Coating Solution: The required volume of an isopropyl alcohol/purified water mixture was transferred into a stainless-steel vessel. Opadry clear (HPMC) or methacrylic copolymers were dispersed therein under stirring until a uniform solution was obtained.
3. Addition of Buffering Agents: Citric acid monohydrate and sodium citrate were slowly added to the Step 2 mixture under stirring to obtain a uniform solution.
4. Addition of Hydrochlorothiazide: Hydrochlorothiazide was slowly added to the Step 3 solution under continuous stirring until a uniform drug-containing coating solution was obtained.
5. Prewarming: The sifted spheres of Step 1 were loaded into a fluid bed processor and prewarmed at a product temperature of 30° C. for 10±5 minutes.
6. Bottom Spray Coating: The prewarmed spheres of Step 5 were coated with the coating solution of Step 4 using a bottom-spray (Wurster) technique until the targeted weight gain was achieved.
7. Sifting: The coated granules were sifted and collected separately.
8. Sifting of Additional Excipients: Sodium benzoate, disodium EDTA, microcrystalline cellulose, sodium carboxymethylcellulose, xanthan gum, peppermint flavor, and crospovidone or colloidal silicon dioxide were sifted and collected separately.
9. Blending: The coated granules from Step 7 were transferred into a blender. The sifted excipients from Step 8 were added and the mixture was blended.
10. Filling: The Lisinopril-part granules and the Hydrochlorothiazide-part granules were combined and filled into sachets.

### Example 5: Benazepril+HCTZ Oral Solution

| Ingredients | Function | % W/W |
|---|---|---|
| Benazepril HCl | Active pharmaceutical | 0.1-2 |
| | ingredient | |
| Hydrochlorothiazide | Active pharmaceutical | 0.15-2.6 |
| | ingredient | |
| Sodium benzoate/Parabens | Preservative | 0.05-2 |
| Xylitol/Mannitol/ | Sweetening agent | 0.5-50 |
| Sorbitol/Sucralose | | |
| Glycerol | Cosolvent | 10-80 |
| Citric acid | Buffering agent | 0.05-5 |
| Sodium citrate | Buffering agent | 0.1-5 |
| Hydroxy | Stabilizing agent | 0.5-30 |
| β-cyclodextrin/Povidone | | |
| Propylene glycol | Cosolvent | 5 -30 |
| Flavor | Flavoring agent | 0.05-3 |
| HCl/NaoH | pH modifier | q.s. |
| Purified water | Solvent | q.s. |

### General Manufacturing Process for Oral Solution

An oral liquid pharmaceutical solution was prepared according to the following procedure:
1. Preparation of Cyclodextrin Solution: Hydroxy-β-cyclodextrin was added to purified water in a separate vessel and mixed until completely dissolved to obtain a clear solution.
2. Addition of Hydrochlorothiazide: Hydrochlorothiazide was added to the Step 1 solution and mixed until fully dissolved, yielding a clear solution.
3. Addition of Preservative: Sodium benzoate was added to the Step 2 solution and mixed until completely dissolved to form a clear solution.
4. Addition of Co-solvents: Glycerol and propylene glycol were added to the Step 3 solution and mixed until uniformly incorporated to provide a clear solution.
5. Addition of Benazepril Hydrochloride: Benazepril hydrochloride was added to the Step 4 solution and mixed until completely dissolved, yielding a clear solution.
6. Addition of Sweeteners: Xylitol, mannitol, or sorbitol was added to the Step 5 solution under stirring and mixed until fully dissolved to obtain a clear solution.
7. Addition of Buffering Agents and Flavor: Citric acid, sodium citrate, and flavoring agents were added to the Step 6 solution under stirring and mixed until completely homogeneous.
8. Volume Adjustment: Purified water was added to the Step 7 solution to bring the mixture to the final desired volume.
9. Filling: The resulting clear oral solution was filled into bottles.

### Example 6: Benazepril+HCTZ Powder/Pellet for Oral Solution

| Ingredients | Function | % W/W |
|---|---|---|
| Benazepril HCl | Active pharmaceutical | 0.1-2 |
| | ingredient | |
| Hydrochlorothiazide | Active pharmaceutical | 0.15-2.6 |
| | ingredient | |
| Sodium benzoate/Parabens | Preservative | 0.05-2 |
| Xylitol/Mannitol/ | Sweetening agent | 0.5-70 |
| Sorbitol/Sucralose | | |
| Citric acid | Buffering agent | 0.05-5 |
| Sodium citrate | Buffering agent | 0.1-5 |
| Hydroxy | Stabilizing agent | 0.5-30 |
| β-cyclodextrin/Povidone | | |
| Flavor | Flavoring agent | 0.05-3 |
| HPMC | Viscosity modifier | 1-15 |
| Xanthan gum | Viscosity modifier | 0.05-7 |
| Hydroxypropyl | Coating agent | 0.5-15 |
| cellulose/HPMC/ | | |
| methacrylic copolymers | | |
| Opadry clear | Coating agent | 0.5-15 |
| (HPMC based) | | |
| Sugar/Mannitol spheres | Inert pellets | 1-15 |
| Isopropyl alcohol | Solvent | q.s. |
| Purified water | Solvent | q.s. |

### General Manufacturing Process of Powder/Pellet for Oral Solution

### Preparation of the Benazepril HCl Part:

1. Sifting of Dry Materials: Benazepril hydrochloride, maltitol or mannitol, and sodium benzoate were sifted and transferred into a rapid mixer granulator (RMG).
2. Preparation of Binder Solution: The required quantity of an isopropyl alcohol/purified water mixture was dispensed into a stainless-steel vessel. Xanthan gum was dispersed therein under stirring until a uniform, lump-free dispersion was obtained.
3. Initial Mixing: The sifted materials of Step 1 were mixed in the RMG for 10 minutes with the impeller on and the chopper off.
4. Addition of Binder Solution: The binder solution of Step 2 was added to the Step 3 mixture with the impeller on and the chopper off.
5. Kneading: The resulting wet mass was kneaded with both the impeller and chopper on to form granules.
6. Optional Moisture Adjustment: If required, additional isopropyl alcohol/purified water mixture was added to the wet mass of Step 5 with the impeller on and chopper off, followed by further kneading with both impeller and chopper on.
7. Drying: The wet mass of Step 6 was transferred to a fluid bed dryer (FBD) and dried at a low temperature until a loss on drying (LOD) of not more than 2.0% w/w was achieved.
8. Milling: The dried granules obtained in Step 7 were milled and sifted using a milling and sifting machine, and the milled granules were collected.

### Preparation of the Hydrochlorothiazide Part—by Top Spray Technique:

1. Sifting of Dry Materials: Hydrochlorothiazide, maltitol or mannitol, and hydroxy-β-cyclodextrin or povidone were sifted.
2. Preparation of Coating Solution: The required quantity of an isopropyl alcohol/water mixture was transferred into a stainless-steel vessel. Opadry clear (HPMC) or methacrylic copolymers were dispersed therein under stirring until a uniform solution was obtained.
3. Prewarming: The sifted materials of Step 1 were loaded into a fluid bed processor and prewarmed to a product temperature of 30° C. for 10±5 minutes.
4. Top Spray Coating: The materials from Step 3 were coated with the solution of Step 2 using a top-spray technique until the desired weight gain was achieved.
5. Sifting: The coated granules were sifted and collected separately.
6. Sifting of Additional Excipients: Hydroxypropyl cellulose, citric acid, sodium citrate, and flavoring agents were sifted and collected separately.
7. Blending: The coated granules from Step 5 were transferred to a blender. The sifted excipients of Step 6 were added, and the mixture was blended until homogeneous.

### Preparation of the Hydrochlorothiazide Part—by Wurster Technique:

1. Sifting of Starter Cores: Sugar spheres or mannitol spheres were sifted.
2. Preparation of Coating Solution: The required quantity of an isopropyl alcohol/purified water mixture was dispensed into a stainless-steel vessel. Opadry clear (HPMC) or methacrylic copolymers were dispersed therein under stirring until a uniform solution was obtained.
3. Addition of Drug: Hydrochlorothiazide was slowly added to the Step 2 mixture under stirring until a uniform drug-containing coating solution was formed.
4. Prewarming: The starter spheres of Step 1 were loaded into a fluid bed processor and prewarmed to a product temperature of 30° C. for 10±5 minutes.
5. Bottom Spray Coating: The prewarmed spheres were coated with the coating solution of Step 3 using a bottom-spray (Wurster) technique until the desired weight gain was achieved.
6. Sifting: The coated granules were sifted and collected separately.
7. Sifting of Additional Excipients: Hydroxypropyl cellulose, citric acid, sodium citrate, and flavoring agents were sifted and collected separately.
8. Blending: The coated granules from Step 6 were transferred into a blender. The sifted excipients from Step 7 were added, and the mixture was blended.
9. Filling: The granules of the Benazepril hydrochloride part and the granules of the Hydrochlorothiazide part were combined and filled into sachets.

### Example 7: Benazepril+HCTZ Oral Suspension

| Ingredients | Function | % W/W |
|---|---|---|
| Benazepril HCl | Active pharmaceutical | 0.1-2 |
| | ingredient | |
| Hydrochlorothiazide | Active pharmaceutical | 0.15-2.6 |
| | ingredient | |
| Sodium benzoate/Parabens | Preservative | 0.05-2 |
| Xylitol/Mannitol/ | Sweetening agent | 0.5-50 |
| Sorbitol/Sucralose | | |
| Glycerol | Cosolvent | 10-80 |
| Citric acid | Buffering agent | 0.05-5 |
| Sodium citrate | Buffering agent | 0.1-5 |
| Hydroxy | Stabilizing agent | 0.5-30 |
| β-cyclodextrin/Povidone | | |
| Propylene glycol | Cosolvent | 5-30 |
| Hydroxyethyl | Viscosity modifier | 0.05-7 |
| cellulose/Xanthan gum | | |
| Flavour | Flavoring agent | 0.05-3 |
| HCl/NaoH | pH modifier | q.s. |
| Purified water | Solvent | q.s. |
| Microcrystalline cellulose | Viscosity modifier | 0.2-15 |
| Carboxymethylcellulose | Viscosity modifier | 0.05-10 |
| sodium | | |
| Crospovidone/Colloidal | Anticaking agent | 0.05-10 |
| silicon dioxide | | |

### General Manufacturing Process for Oral Suspension

An oral pharmaceutical suspension was prepared as follows:
1. Preparation of Cyclodextrin Solution: Hydroxy-β-cyclodextrin was added to purified water or isopropyl alcohol in a separate vessel and mixed until completely dissolved to obtain a clear solution.
2. Addition of Hydrochlorothiazide: Hydrochlorothiazide was added to the Step 1 solution and mixed until fully dissolved, yielding a clear solution.
3. Addition of Preservative: Sodium benzoate was added to the Step 2 solution and mixed until dissolved to form a clear solution.
4. Addition of Co-solvent: Propylene glycol was added to the Step 3 solution and mixed until uniformly incorporated to provide a clear solution.
5. Preparation of Benazepril Hydrochloride Solution: Benazepril hydrochloride was added to purified water in a separate vessel and mixed until completely dissolved to yield a clear drug solution.
6. Preparation of Polymer Solution: Hydroxyethyl cellulose or xanthan gum was added to glycerol in a separate vessel and mixed until completely dissolved to obtain a clear polymer solution.
7. Combination of Polymer and Cyclodextrin Mixtures: The polymer solution of Step 6 was added to the Step 4 mixture under stirring and mixed until homogeneous.
8. Addition of Sweeteners and Suspending Agents: Maltitol, mannitol, or sucralose, together with microcrystalline cellulose and sodium carboxymethylcellulose, were added to the Step 7 mixture under stirring and mixed until a uniform, lump-free suspension was obtained.
9. Incorporation of Benazepril Hydrochloride Solution: The drug solution of Step 5 was added to the Step 8 suspension under stirring and mixed until a homogeneous, lump-free suspension was formed.
10. Addition of Buffering Agents and Additional Excipients: Citric acid, sodium citrate, flavoring agents, and crospovidone or colloidal silicon dioxide were added to the Step 9 suspension under stirring and mixed until uniformly incorporated.
11. Volume Adjustment: Purified water was added to the Step 10 suspension to bring the mixture to the final desired volume.
12. Filling: The resulting suspension was filled into bottles.

### Example 8: Benazepril+HCTZ Powder/Pellet for Oral Suspension

| Ingredients | Function | % W/W |
|---|---|---|
| Benazepril HCl | Active pharmaceutical | 0.1-2 |
| | ingredient | |
| Hydrochlorothiazide | Active pharmaceutical | 0.15-2.6 |
| | ingredient | |
| Sodium benzoate | Preservative | 0.05-2 |
| Xylitol/Mannitol/ | Sweetening agent | 0.5-70 |
| Sorbitol/Sucralose | | |
| Citric acid | Buffering agent | 0.05-5 |
| Sodium citrate | Buffering agent | 0.1-5 |
| Hydroxyethyl | Viscosity modifier | 0.05-7 |
| cellulose/Xanthan gum | | |
| Flavor | Flavoring agent | 0.05-3 |
| Purified water | Solvent | q.s. |
| Microcrystalline cellulose | Viscosity modifier | 0.2-15 |
| Carboxymethylcellulose | Viscosity modifier | 0.05-10 |
| sodium | | |
| Crospovidone/Colloidal | Anticaking agent | 0.05-10 |
| silicon dioxide | | |
| Hydroxyethyl cellulose | Viscosity modifier | 0.1-5 |
| HPMC | Viscosity modifier | 1-15 |
| Hydroxypropyl | Coating agent | 0.5-15 |
| cellulose/HPMC/ | | |
| methacrylic copolymers | | |
| Opadry clear (HPMC based) | Coating agent | 0.5-15 |
| Sugar/Mannitol spheres | Inert pellets | 1-15 |
| Isopropyl alcohol | Solvent | q.s. |

### General Manufacturing Process of Powder/Pellet for Oral Suspension

### A. Preparation of the Benazepril HCl Part:

1. Sifting of Dry Materials: Benazepril hydrochloride, maltitol or mannitol, and sodium benzoate were sifted and transferred into a rapid mixer granulator (RMG).
2. Preparation of Binder Solution: The required quantity of an isopropyl alcohol/purified water mixture was dispensed into a stainless-steel vessel. Xanthan gum was dispersed therein under stirring until a uniform, lump-free dispersion was obtained.
3. Initial Mixing: The sifted materials of Step 1 were mixed in the RMG for 10 minutes with the impeller on and the chopper off.
4. Addition of Binder: The binder solution of Step 2 was added to the mixture of Step 3 with the impeller on and the chopper off.
5. Kneading: The resulting wet mass was kneaded with both the impeller and chopper on to form granules.
6. Optional Moisture Adjustment: If required, additional isopropyl alcohol/purified water mixture was added to the Step 5 wet mass with the impeller on and the chopper off, followed by kneading with both impeller and chopper on.
7. Drying: The wet mass from Step 6 was transferred to a fluid bed dryer (FBD) and dried at a low temperature until a loss on drying (LOD) of not more than 2.0% w/w was achieved.
8. Milling: The dried granules obtained in Step 7 were milled and sifted using a milling and sifting machine, and the resulting granules were collected.

### B. Preparation of the Hydrochlorothiazide Part—by Top Spray Technique:

1. Sifting of Dry Materials: Hydrochlorothiazide and maltitol or mannitol were sifted.
2. Preparation of Coating Solution: The required quantity of an isopropyl alcohol/water mixture was transferred to a stainless-steel vessel. Opadry clear (HPMC) or methacrylic copolymers were dispersed under stirring until a uniform solution was formed.
3. Prewarming: The sifted materials from Step 1 were loaded into a fluid bed processor and prewarmed to a product temperature of 30° C. for 10±5 minutes.
4. Top Spray Coating: The prewarmed materials of Step 3 were coated with the solution of Step 2 using a top-spray technique until the desired weight gain was achieved.
5. Sifting: The coated granules were sifted and collected separately.
6. Sifting of Additional Excipients: Hydroxypropyl cellulose, citric acid, sodium citrate, microcrystalline cellulose, sodium carboxymethylcellulose, and flavoring agents were sifted and collected separately.
7. Blending: The coated granules of Step 5 were transferred to a blender. The sifted excipients from Step 6 were added, and the mixture was blended until homogeneous.

### C. Preparation of the Hydrochlorothiazide Part—by Wurster Technique:

1. Sifting of Starter Cores: Sugar spheres or mannitol spheres were sifted.
2. Preparation of Coating Solution (Polymer Phase): The required volume of an isopropyl alcohol/purified water mixture was dispensed into a stainless-steel vessel. Opadry clear (HPMC) or methacrylic copolymers were slowly dispersed therein under stirring until a uniform solution was obtained.
3. Addition of Buffering Agents: Citric acid and sodium citrate were slowly added to the Step 2 mixture under stirring to obtain a uniform buffering solution.
4. Addition of Hydrochlorothiazide: Hydrochlorothiazide was slowly added to the Step 3 solution under stirring until a uniform drug-containing coating solution was obtained.
5. Prewarming: The sifted spheres of Step 1 were loaded into a fluid bed processor and prewarmed to a product temperature of 30° C. for 10±5 minutes.
6. Bottom Spray Coating: The spheres from Step 5 were coated with the Step 4 coating solution using a bottom-spray (Wurster) technique until the desired weight gain was achieved.
7. Sifting: The coated granules were sifted and collected separately.
8. Sifting of Additional Excipients: Sodium benzoate, microcrystalline cellulose, sodium carboxymethylcellulose, xanthan gum, flavoring agents, and crospovidone or colloidal silicon dioxide were sifted and collected separately.
9. Blending: The coated granules of Step 7 were transferred into a blender. The sifted excipients of Step 8 were added, and the mixture was blended.
10. Filling: The granules of the Benazepril hydrochloride part and the granules of the Hydrochlorothiazide part were combined and filled into sachets.

### Example 9: Benazepril Oral Solution

| Ingredients | Function | % W/W |
|---|---|---|
| Benazepril HCl | Active pharmaceutical | 0.1-2 |
| | ingredient | |
| Sodium benzoate/Parabens | Preservative | 0.05-2 |
| Xylitol/Mannitol/ | Sweetening agent | 0.5-50 |
| Sorbitol/Sucralose | | |
| Glycerol | Cosolvent | 10-80 |
| Citric acid | Buffering agent | 0.05-5 |
| Sodium citrate | Buffering agent | 0.1-5 |
| Hydroxy | Stabilizing agent | 0.5-30 |
| β-cyclodextrin/Povidone | | |
| Propylene glycol | Cosolvent | 5-30 |
| Flavor | Flavoring agent | 0.05-3 |
| HCl/NaoH | pH modifier | q.s. |
| Purified water | Solvent | q.s. |

### General Manufacturing Process for Oral Solution

An oral pharmaceutical solution was prepared as follows:
1. Preparation of Cyclodextrin Solution: Hydroxy-β-cyclodextrin was added to purified water in a separate vessel and mixed until completely dissolved to obtain a clear solution.
2. Addition of Benazepril Hydrochloride: Benazepril hydrochloride was added to the Step 1 solution and mixed until fully dissolved, yielding a clear solution.
3. Addition of Preservative: Sodium benzoate was added to the Step 2 solution and mixed until completely dissolved to form a clear solution.
4. Addition of Co-solvents: Glycerol and propylene glycol were added to the Step 3 solution and mixed until uniformly incorporated to provide a clear solution.
5. Addition of Sweeteners: Xylitol, mannitol, or sorbitol was added to the Step 4 mixture under stirring and mixed until fully dissolved to obtain a clear solution.
6. Addition of Buffering Agents and Flavor: Citric acid, sodium citrate, and flavoring agents were added to the Step 5 solution under stirring and mixed until completely homogeneous.
7. Volume Adjustment: Purified water was added to the Step 6 mixture to bring the solution to the final desired volume.
8. Filling: The resulting clear solution was filled into bottles.

### Example 10: Benazepril Powder/Pellet for Oral Solution

| Ingredients | Function | % W/W |
|---|---|---|
| Benazepril HCl | Active pharmaceutical | 0.1-2 |
| | ingredient | |
| Sodium benzoate/Parabens | Preservative | 0.05-2 |
| Xylitol/Mannitol/ | Sweetening agent | 0.5-70 |
| Sorbitol/Sucralose | | |
| Citric acid | Buffering agent | 0.05-5 |
| Sodium citrate | Buffering agent | 0.1-5 |
| Hydroxy | Stabilizing agent | 0.5-30 |
| β-cyclodextrin/Povidone | | |
| Flavor | Flavoring agent | 0.05-3 |
| HPMC | Viscosity modifier | 1-15 |
| Xanthan gum | Viscosity modifier | 0.05-7 |
| Hydroxypropyl | Coating agent | 0.5-15 |
| cellulose/HPMC/ | | |
| methacrylic copolymers | | |
| Opadry clear | Coating agent | 0.5-15 |
| (HPMC based) | | |
| Sugar/Mannitol spheres | Inert pellets | 1-15 |
| Isopropyl alcohol | Solvent | q.s. |
| Purified water | Solvent | q.s. |

### General Manufacturing Process of Powder/Pellet for Oral Solution

### A. Preparation of the Benazepril HCl—by Top Spray Technique:

1. Sifting of Dry Materials: Benazepril hydrochloride, maltitol or mannitol, and hydroxy-β-cyclodextrin or povidone were sifted.
2. Preparation of Coating Solution: The required quantity of an isopropyl alcohol/water mixture was transferred into a stainless-steel vessel. Opadry clear (HPMC) or methacrylic copolymers were slowly dispersed under stirring until a uniform solution was obtained.
3. Prewarming: The sifted materials from Step 1 were loaded into a fluid bed processor and prewarmed to a product temperature of 30° C. for 10±5 minutes.
4. Top Spray Coating: The prewarmed materials from Step 3 were coated with the coating solution of Step 2 using a top-spray technique until the desired weight gain was achieved.
5. Sifting: The coated granules were sifted and collected separately.
6. Sifting of Additional Excipients: Xanthan gum, sodium benzoate, hydroxypropyl cellulose, citric acid, sodium citrate, and flavoring agents were sifted and collected separately.
7. Blending: The coated granules of Step 5 were transferred to a blender. The sifted excipients of Step 6 were added, and the mixture was blended to obtain a uniform granulate.

### B. Preparation of the Benazepril HCl—by Wurster Technique:

1. Sifting of Starter Cores: Sugar spheres or mannitol spheres were sifted.
2. Preparation of Coating Solution (Polymer Phase): The required quantity of an isopropyl alcohol/purified water mixture was transferred to a stainless-steel vessel. Opadry clear (HPMC) or methacrylic copolymers were slowly dispersed under stirring until a uniform solution was obtained.
3. Addition of Drug: Hydrochlorothiazide was slowly added to the Step 2 solution under continuous stirring until a uniform drug-containing coating solution was formed.
4. Prewarming: The sifted spheres of Step 1 were loaded into a fluid bed processor and prewarmed to a product temperature of 30° C. for 10±5 minutes.
5. Bottom Spray Coating: The prewarmed spheres from Step 4 were coated with the Step 3 coating solution using a bottom-spray (Wurster) technique until the desired weight gain was achieved.
6. Sifting: The coated granules were sifted and collected separately.
7. Sifting of Additional Excipients: Xanthan gum, hydroxypropyl cellulose, citric acid, sodium citrate, and flavoring agents were sifted and collected separately.
8. Blending: The coated granules of Step 6 were transferred to a blender. The sifted excipients of Step 7 were added, and the mixture was blended to obtain a uniform granulate.
9. Filling: The Benazepril hydrochloride granules prepared by either technique were filled into sachets.

### Example 11: Benazepril Oral Suspension

| Ingredients | Function | % W/W |
|---|---|---|
| Benazepril HCl | Active pharmaceutical | 0.1-2 |
| | ingredient | |
| Sodium benzoate/Parabens | Preservative | 0.05-2 |
| Xylitol/Mannitol/ | Sweetening agent | 0.5-50 |
| Sorbitol/Sucralose | | |
| Glycerol | Cosolvent | 10-80 |
| Citric acid | Buffering agent | 0.05-5 |
| Sodium citrate | Buffering agent | 0.1-5 |
| Hydroxy | Stabilizing agent | 0.5-30 |
| β-cyclodextrin/Povidone | | |
| Propylene glycol | Cosolvent | 5-30 |
| Hydroxyethyl | Viscosity modifier | 0.05-7 |
| cellulose/Xanthan gum | | |
| Flavour | Flavoring agent | 0.05-3 |
| HCl/NaoH | pH modifier | q.s. |
| Purified water | Solvent | q.s. |
| Microcrystalline cellulose | Viscosity modifier | 0.2-15 |
| Carboxymethylcellulose | Viscosity modifier | 0.05-10 |
| sodium | | |
| Crospovidone/Colloidal | Anticaking agent | 0.05-10 |
| silicon dioxide | | |

### General Manufacturing Process for Oral Suspension

An oral pharmaceutical suspension was prepared according to the following procedure:
1. Preparation of Cyclodextrin Solution: Hydroxy-β-cyclodextrin was added to purified water or isopropyl alcohol in a separate vessel and mixed until completely dissolved to obtain a clear solution.
2. Addition of Benazepril Hydrochloride: Benazepril hydrochloride was added to the Step 1 solution and mixed until fully dissolved, yielding a clear solution.
3. Addition of Preservative: Sodium benzoate was added to the Step 2 mixture and mixed until completely dissolved to form a clear solution.
4. Addition of Co-solvent: Propylene glycol was added to the Step 3 solution and mixed until uniformly incorporated.
5. Preparation of Polymer Solution: Hydroxyethyl cellulose or xanthan gum was added to glycerol in a separate vessel and mixed until completely dissolved to provide a clear polymer solution.
6. Combination of Polymer and Drug-Carrier Mixtures: The Step 5 polymer solution was added to the Step 4 solution under stirring and mixed until homogeneous.
7. Addition of Sweeteners and Suspending Agents: Maltitol, mannitol, or sucralose, along with microcrystalline cellulose and sodium carboxymethylcellulose, were added to the Step 6 mixture under stirring and mixed until a uniform, lump-free suspension was formed.
8. Addition of Buffering Agents and Additional Excipients: Citric acid, sodium citrate, flavoring agents, and crospovidone or colloidal silicon dioxide were added to the Step 7 suspension under stirring and mixed until homogeneous and lump-free.
9. Volume Adjustment: Purified water was added to the Step 8 suspension to bring the mixture to the final desired volume.
10. Filling: The resulting oral suspension was filled into bottles.

### Example 12: Benazepril Powder/Pellet for Oral Suspension

| Ingredients | Function | % W/W |
|---|---|---|
| Benazepril HCl | Active pharmaceutical | 0.1-2 |
| | ingredient | |
| Sodium benzoate | Preservative | 0.05-2 |
| Xylitol/Mannitol/ | Sweetening agent | 0.5-70 |
| Sorbitol/Sucralose | | |
| Citric acid | Buffering agent | 0.05-5 |
| Sodium citrate | Buffering agent | 0.1-5 |
| Hydroxyethyl | Viscosity modifier | 0.05-7 |
| cellulose/Xanthan gum | | |
| Flavor | Flavoring agent | 0.05-3 |
| Purified water | Solvent | q.s. |
| Microcrystalline cellulose | Viscosity modifier | 0.2-15 |
| Carboxymethylcellulose | Viscosity modifier | 0.05-10 |
| sodium | | |
| Crospovidone/Colloidal | Anticaking agent | 0.05-10 |
| silicon dioxide | | |
| Hydroxyethyl cellulose | Viscosity modifier | 0.1-5 |
| HPMC | Viscosity modifier | 1-15 |
| Hydroxypropyl | Coating agent | 0.5-15 |
| cellulose/HPMC/ | | |
| methacrylic copolymers | | |
| Opadry clear | Coating agent | 0.5-15 |
| (HPMC based) | | |
| Sugar/Mannitol spheres | Inert pellets | 1-15 |
| Isopropyl alcohol | Solvent | q.s. |

### General Manufacturing Process of Powder/Pellet for Oral Suspension

### A. Preparation of the Benazepril HCl—by Top Spray Technique:

1. Sifting of Dry Materials: Benazepril hydrochloride and mannitol were sifted.
2. Preparation of Coating Solution: The weighed quantity of an isopropyl alcohol/water mixture was transferred into a stainless-steel vessel. Opadry clear (HPMC) or methacrylic copolymers were slowly dispersed therein under stirring until a uniform solution was obtained.
3. Prewarming: The sifted materials from Step 1 were loaded into a fluid bed processor and prewarmed to a product temperature of 30° C. for 10±5 minutes.
4. Top Spray Coating: The prewarmed materials of Step 3 were coated with the coating solution of Step 2 using a top-spray technique until the desired weight gain was achieved.
5. Sifting: The coated granules were sifted and collected separately.
6. Sifting of Additional Excipients: Sodium benzoate, xanthan gum, hydroxypropyl cellulose, citric acid, sodium citrate, microcrystalline cellulose, sodium carboxymethylcellulose, and flavoring agents were sifted and collected separately.
7. Blending: The coated granules from Step 5 were transferred to a blender. The sifted excipients from Step 6 were added, and the mixture was blended until uniform.

### B. Preparation of the Benazepril HCl—by Wurster Technique:

1. Sifting of Starter Cores: Sugar spheres or mannitol spheres were sifted.
2. Preparation of Polymer Solution: The required quantity of an isopropyl alcohol/purified water mixture was transferred into a stainless-steel vessel. Opadry clear (HPMC) or methacrylic copolymers were slowly dispersed under stirring until a uniform solution was obtained.
3. Addition of Buffering Agents: Citric acid and sodium citrate were slowly added to the Step 2 solution under stirring until a homogeneous solution was obtained.
4. Addition of Benazepril Hydrochloride: Benazepril hydrochloride was slowly added to the Step 3 mixture under stirring and mixed until a uniform drug-containing coating solution was formed.
5. Prewarming: The spheres from Step 1 were loaded into a fluid bed processor and prewarmed to a product temperature of 30° C. for 10±5 minutes.
6. Bottom Spray Coating: The Step 5 spheres were coated with the drug-containing coating solution of Step 4 using a bottom-spray (Wurster) technique until the desired weight gain was achieved.
7. Sifting: The coated granules were sifted and collected separately.
8. Sifting of Additional Excipients: Sodium benzoate, microcrystalline cellulose, sodium carboxymethylcellulose, xanthan gum, flavoring agents, and crospovidone or colloidal silicon dioxide were sifted and collected separately.
9. Blending: The coated granules from Step 7 were transferred into a blender. The sifted excipients of Step 8 were added, and the mixture was blended until uniform.
10. Filling: The Benazepril hydrochloride granules produced by either the top-spray or Wurster process were filled into sachets.

### Example 13: HCTZ Oral Suspension

| Ingredients | Function | % W/W |
|---|---|---|
| Hydrochlorothiazide | Active pharmaceutical | 0.15-2.6 |
| | ingredient | |
| Sodium benzoate/Parabens | Preservative | 0.05-2 |
| Xylitol/Mannitol/ | Sweetening agent | 0.5-50 |
| Sorbitol/Sucralose | | |
| Glycerol | Cosolvent | 10-80 |
| Citric acid | Buffering agent | 0.05-5 |
| Sodium citrate | Buffering agent | 0.1-5 |
| Hydroxy | Stabilizing agent | 0.5-30 |
| β-cyclodextrin/Povidone | | |
| Propylene glycol | Cosolvent | 5-30 |
| Hydroxyethyl | Viscosity modifier | 0.05-7 |
| cellulose/Xanthan gum | | |
| Flavour | Flavoring agent | 0.05-3 |
| HCl/NaoH | pH modifier | q.s. |
| Purified water | Solvent | q.s. |
| Microcrystalline cellulose | Viscosity modifier | 0.2-15 |
| Carboxymethylcellulose | Viscosity modifier | 0.05-10 |
| sodium | | |
| Crospovidone/Colloidal | Anticaking agent | 0.05-10 |
| silicon dioxide | | |

### General Manufacturing Process for Oral Suspension

An oral pharmaceutical suspension was prepared according to the following procedure:
1. Preparation of Cyclodextrin Solution: Hydroxy-β-cyclodextrin was added to purified water or isopropyl alcohol in a separate vessel and mixed until completely dissolved to obtain a clear solution.
2. Addition of Hydrochlorothiazide: Hydrochlorothiazide was added to the Step 1 solution and mixed until fully dissolved, yielding a clear solution.
3. Addition of Preservative: Sodium benzoate was added to the Step 2 mixture and mixed until completely dissolved to form a clear solution.
4. Addition of Co-solvent: Propylene glycol was added to the Step 3 solution and mixed until uniformly incorporated.
5. Preparation of Polymer Solution: Hydroxyethyl cellulose or xanthan gum was added to glycerol in a separate vessel and mixed until completely dissolved to obtain a clear polymer solution.
6. Combination of Solutions: The polymer solution of Step 5 was added to the Step 4 mixture under stirring and mixed until uniform.
7. Addition of Sweeteners and Suspending Agents: Maltitol, mannitol, or sucralose, together with microcrystalline cellulose and sodium carboxymethylcellulose, were added to the Step 6 mixture under stirring and mixed until a lump-free suspension was obtained.
8. Addition of Buffering Agents and Additional Excipients: Citric acid, sodium citrate, flavoring agents, and crospovidone or colloidal silicon dioxide were added to the Step 7 suspension under stirring and mixed until uniformly incorporated and lump-free.
9. Volume Adjustment: Purified water was added to the Step 8 suspension to bring the mixture to the final required volume.
10. Filling: The resulting oral suspension was filled into bottles.

### Example 14: HCTZ Powder/Pellet for Oral Suspension

| Ingredients | Function | % W/W |
|---|---|---|
| Hydrochlorothiazide | Active pharmaceutical | 0.15-2.6 |
| | ingredient | |
| Sodium benzoate | Preservative | 0.05-2 |
| Xylitol/Mannitol/ | Sweetening agent | 0.5-70 |
| Sorbitol/Sucralose | | |
| Citric acid | Buffering agent | 0.05-5 |
| Sodium citrate | Buffering agent | 0.1-5 |
| Hydroxyethyl | Viscosity modifier | 0.05-7 |
| cellulose/Xanthan gum | | |
| Flavor | Flavoring agent | 0.05-3 |
| Purified water | Solvent | q.s. |
| Microcrystalline cellulose | Viscosity modifier | 0.2-15 |
| Carboxymethylcellulose | Viscosity modifier | 0.05-10 |
| sodium | | |
| Crospovidone/Colloidal | Anticaking agent | 0.05-10 |
| silicon dioxide | | |
| Hydroxyethyl cellulose | Viscosity modifier | 0.1-5 |
| HPMC | Viscosity modifier | 1-15 |
| Hydroxypropyl | Coating agent | 0.5-15 |
| cellulose/HPMC/ | | |
| methacrylic copolymers | | |
| Opadry clear | Coating agent | 0.5-15 |
| (HPMC based) | | |
| Sugar/Mannitol spheres | Inert pellets | 1-15 |
| Isopropyl alcohol | Solvent | q.s. |

### General Manufacturing Process of Powder/Pellet for Oral Suspension

### A. Preparation of the Hydrochlorothiazide—by Top Spray Technique:

1. Sifting of Dry Materials: Hydrochlorothiazide and mannitol were sifted.
2. Preparation of Coating Solution: The required quantity of an isopropyl alcohol/water mixture was transferred into a stainless-steel vessel. Opadry clear (HPMC) or methacrylic copolymers were slowly dispersed under stirring until a uniform solution was obtained.
3. Prewarming: The sifted materials from Step 1 were loaded into a fluid bed processor and prewarmed to a product temperature of 30° C. for 10±5 minutes.
4. Top Spray Coating: The prewarmed materials were coated with the coating solution of Step 2 using a top-spray technique until the desired weight gain was achieved.
5. Sifting: The coated granules were sifted and collected separately.
6. Sifting of Additional Excipients: Sodium benzoate, xanthan gum, hydroxypropyl cellulose, citric acid, sodium citrate, microcrystalline cellulose, sodium carboxymethylcellulose, crospovidone or colloidal silicon dioxide, and flavoring agents were sifted and collected separately.
7. Blending: The coated granules from Step 5 were transferred to a blender. The sifted excipients of Step 6 were added, and the mixture was blended until uniform.

### B. Preparation of the Hydrochlorothiazide—by Wurster Technique:

1. Sifting of Starter Cores: Sugar spheres or mannitol spheres were sifted.
2. Preparation of Polymer Solution: The required quantity of an isopropyl alcohol/purified water mixture was transferred into a stainless-steel vessel. Opadry clear (HPMC) or methacrylic copolymers were slowly dispersed under stirring until a uniform solution was obtained.
3. Addition of Buffering Agents: Citric acid and sodium citrate were slowly added to the Step 2 solution under stirring until a homogeneous solution was formed.
4. Addition of Hydrochlorothiazide: Hydrochlorothiazide was slowly added to the Step 3 mixture under stirring and mixed until a uniform drug-containing coating solution was obtained.
5. Prewarming: The spheres from Step 1 were loaded into a fluid bed processor and prewarmed to a product temperature of 30° C. for 10±5 minutes.
6. Bottom Spray Coating: The prewarmed spheres were coated with the Step 4 solution using a bottom-spray (Wurster) technique until the desired weight gain was achieved.
7. Sifting: The coated granules were sifted and collected separately.
8. Sifting of Additional Excipients: Sodium benzoate, microcrystalline cellulose, sodium carboxymethylcellulose, xanthan gum, flavoring agents, and crospovidone or colloidal silicon dioxide were sifted and collected separately.
9. Blending: The coated granules of Step 7 were transferred into a blender. The sifted excipients of Step 8 were added, and the mixture was blended until uniform.
10. Filling: The Hydrochlorothiazide granules prepared by either technique were filled into sachets.

### Example 15: Benazepril Hydrochloride Powder for Oral Solution

| Ingredients | Function | % W/W |
|---|---|---|
| Benazepril Hydrochloride | Active pharmaceutical | 0.1-2 |
| | ingredient | |
| Citric Acid Monohydrate | Buffering Agent | 0.05-5 |
| Disodium Edetate | Stabilizing Agent | 0.5-30 |
| Maltitol Powder | Sweetening agent | 0.5-70 |
| Sucralose | Sweetening agent | |
| Colloidal silicon dioxide | Anticaking agent | 0.05-15 |
| Peppermint Flavour | Flavoring agent | 0.05-3 |
| Purified water | Vehicle | Q.s |
| (for Reconstitution) | | |

### Example 16: Lisinopril+HCTZ Powder for Oral Suspension

| Ingredients | Function | % W/W |
|---|---|---|
| Lisinopril Dihydrate | Active pharmaceutical | 0.1-2 |
| | ingredient | |
| Hydrochlorothiazide | Active pharmaceutical | 0.15-2.6 |
| | ingredient | |
| Sodium benzoate | Preservative | 0.05-2 |
| Citric Acid Monohydrate | Buffering Agent | 0.05-5 |
| Carboxymethylcellulose | Viscosity modifiers/suspension | 0.05-15 |
| sodium | stabilizer | |
| Maltitol | Sweetening agent | 0.5-70 |
| Sucralose | Sweetening agent | |
| Microcrystalline | Viscosity modifiers/suspension | 0.05-15 |
| Cellulose | stabilizer | |
| Xanthan Gum | Suspension stabilizer/Viscosity | 0.05-15 |
| | modifiers | |
| Colloidal silicon dioxide | Anticaking agent | 0.05-15 |
| Peppermint Flavor | Flavoring agent | 0.05-3 |
| Purified water | Vehicle | Q.s |
| (for Reconstitution) | | |

### Example 17: Benazepril Hydrochloride+HCTZ Powder for Oral Suspension

| Ingredients | Function | % W/W |
|---|---|---|
| Benazepril HCl | Active pharmaceutical | 0.1-2 |
| | ingredient | |
| Hydrochlorothiazide | Active pharmaceutical | 0.15-2.6 |
| | ingredient | |
| Sodium benzoate | Preservative | 0.05-2 |
| Citric Acid monohydrate | Buffering Agent | 0.05-5 |
| Carboxymethylcellulose | Viscosity modifier/suspension | 0.05-15 |
| sodium | stabilizer | |
| Maltitol | Sweetening agent | 0.5-70 |
| Sucralose | Sweetening agent | |
| Microcrystalline | Viscosity modifier/suspension | 0.05-15 |
| Cellulose | stabilizer | |
| Xanthan Gum | Suspension stabilizer/ | 0.05-15 |
| | Viscosity modifier | |
| Colloidal silicone dioxide | Anticaking agent | 0.05-15 |
| Peppermint Flavor | Flavoring agent | 0.05-3 |
| Purified water | Vehicle | Q.s |
| (for Reconstitution) | | |

### General Manufacturing Procedure Powder for Oral Suspension:

Microcrystalline cellulose 102, xanthan gum, sucralose, peppermint flavor, sodium carboxymethylcellulose, colloidal silicon dioxide, benazepril/hydrochlorothiazide/lisinopril (as applicable), sodium benzoate, citric acid monohydrate, maltitol, sucralose, and disodium edetate were sifted through a #30 mesh screen. The sifted materials were transferred into a blender and blended for 20 minutes. After blending, the resulting powder mixture was unloaded and stored in a double-lined polybag.

### Example 18: HCTZ Oral Suspension

| Ingredients | Function | % W/W |
|---|---|---|
| Hydrochlorothiazide | API | 0.15-2.6 |
| Disodium Edetate | Stabilizing Agent | 0.5-30 |
| Butylated hydroxy | Stabilizing agent/ | 0.01-1.0 |
| anisole | Antioxidant | |
| Citric Acid Monohydrate | Buffering Agent | 0.05-5 |
| Sodium Citrate | Buffering Agent | 0.05-5 |
| Maltitol Liquid | Sweetening agent | 0.05-70 |
| Sucralose | Sweetening agent | |
| Propylene Glycol | Co-solvent | 5-30 |
| Refined Glycerin | Solvent | Q.s |
| Strawberry Flavor | Flavoring agent | 0.05-3 |
| Colloidal silicon | Anticaking agent | 0.05-15 |
| dioxide | | |

### General Manufacturing Procedure for Oral Suspension:

1. Preparation of Antioxidant Solution: Propylene glycol (80%) was transferred into a stainless-steel vessel and heated to 50-60° C. The dispensed quantity of butylated hydroxyanisole was added under mixing until completely dissolved to obtain a clear solution. The solution was then allowed to cool to room temperature.
2. Preparation of Chelating Agent Dispersion: In a separate vessel, glycerin (90%) was added, and the dispensed quantity of disodium edetate was incorporated under continuous stirring until fully dispersed (not less than 30 minutes). The solution of Step 1 was then added to the vessel of this step.
3. Addition of Acidifying Agent: The dispensed quantity of citric acid monohydrate was added to the Step 2 mixture under stirring and mixed until completely dissolved (not less than 10 minutes).
4. Addition of Hydrochlorothiazide: Hydrochlorothiazide was added to the Step 3 mixture under continuous stirring and mixed until completely dissolved (not less than 10 minutes).
5. Addition of Buffer, Sweeteners, and Suspending Agent: Sodium citrate, sucralose, maltitol, and colloidal silicon dioxide were added to the Step 4 mixture under stirring and mixed until completely dissolved (not less than 5 minutes).
6. Addition of Flavor: The dispensed quantity of flavoring agent was added under stirring and mixed until uniformly incorporated (not less than 5 minutes).
7. Volume Adjustment: The mixture was brought to the final required volume or weight using glycerin and stirred for not less than 5 minutes.
8. Homogenization and Filling: The mixture of Step 7 was homogenized for 10 minutes, and the resulting oral suspension was filled into bottles.

1. A stable oral pharmaceutical formulation comprising:
at least one of Lisinopril or Benazepril with Hydrochlorothiazide, Benazepril, Hydrochlorothiazide, a pharmaceutically acceptable salt thereof, a hydrate thereof, a solvate thereof, and/or combinations thereof; and
a sweetening agent.

2. The stable oral pharmaceutical formulation according to claim 1, further comprising a stabilizing agent.

3. The stable oral pharmaceutical formulation according to claim 1, further comprising a viscosity modifier.

4. The stable oral pharmaceutical formulation according to claim 1, further comprising a pharmaceutically acceptable excipient.

5. The stable oral pharmaceutical formulation according to claim 1, wherein the Lisinopril, a pharmaceutically acceptable salt thereof, a hydrate thereof, or a solvate thereof is in an amount of from about 0.1% to about 2% w/w.

6. The stable oral pharmaceutical formulation according to claim 1, wherein the Benazepril, a pharmaceutically acceptable salt thereof, a hydrate thereof, or a solvate thereof is in an amount of from about 0.1% to about 2% w/w.

7. The stable oral pharmaceutical formulation according to claim 1, wherein the Hydrochlorothiazide, a pharmaceutically acceptable salt thereof, a hydrate thereof, or a solvate thereof is in an amount of from about 0.15% to about 2.6% w/w.

8. The stable oral pharmaceutical formulation according to claim 1, wherein the sweetening agent is in an amount of from about 0.5% to about 70% w/w.

9. The stable oral pharmaceutical formulation according to claim 2, wherein the stabilizing agent is in an amount of from about 0.01% to about 30% w/w.

10. The stable oral pharmaceutical formulation according to claim 3, wherein the viscosity modifier is in an amount of from about 0.05% to about 15% w/w.

11. The stable oral pharmaceutical formulation according to claim 1, wherein the sweetening agent is selected from the group consisting of glucose, fructose, sucrose, xylitol, tagatose, sucralose, maltitol, isomaltulose, isomalt, lactitol, sorbitol, erythritol, liquid glucose, compressible sugar, confectioner's sugar, dextrose, maltitol solution, a maltitol oligomer, maltose, mannitol, neohesperidin dihydrochalcone, thaumatin, trehalose, magnasweet, xylitol, trehalose, maltodextrin, polydextrose, glycerin, inulin, maltol, acesulfame and its salts, alitame, aspartame, neotame, sodium cyclamate, saccharin and its salts, stevioside, monkfruit, hydrogenated starch hydrolysates, maltitol syrup, high fructose corn syrup, prosweet, invertose, ora-sweet, ora-sweet-SF, flavored syrups, and the like or their combinations of two or more.

12. The stable oral pharmaceutical formulation according to claim 2, wherein the stabilizing agent is selected from the group consisting of poly-L-lysine, ethylenediaminetetraacetic acid, cyclodextrins (unmodified cyclodextrins such as α-cyclodextrin, β-cyclodextrin and γ-cyclodextrin; substituted cyclodextrins such as partly substituted hydroxyalkyl cyclodextrins, such as hydroxypropyl cyclodextrin or dihydroxypropyl cyclodextrin, partly substituted carboxyalkyl cyclodextrins, such as carboxymethyl cyclodextrin, alkali metal salts of partly substituted carboxyalkyl cyclodextrins or partly substituted water-soluble alkyl cyclodextrins, such as C1 to C4 alkyl cyclodextrins; hydroxypropyl-β-cyclodextrins, sulfobutylether-β-cyclodextrins; methylated β-cyclodextrins such as heptakis (2,6-di-O-methyl)-β-cyclodextrin), RAMEB (statistically methylated or randomly-methylated-β-cyclodextrin β-cyclodextrin), TRIMEB (heptakis (2,3,6-tri-O-methyl)-β-cyclodextrin) and CRYSMEB (weakly methylated β-cyclodextrin) and the like), phosphates, zeolites, ammonia, polyvinylpyrrolidone, sodium alginate, gelatin, silicon dioxide and the like or their combinations of two or more.

13. The stable oral pharmaceutical formulation according to claim 3, wherein the viscosity modifier is selected from the group consisting of hydrocolloid gums, cellulose derivatives, hydroxyethyl cellulose, methylcellulose, carboxymethylcellulose, carbomers, polyvinyl alcohols, silicon dioxide, xanthan gum, crospovidone and the like or their combinations of two or more.

14. The stable oral pharmaceutical formulation according to claim 4, wherein the pharmaceutically acceptable excipient is selected from the group consisting of a flavoring agent, a preservative, a buffering agent, a wetting agent, a solvent, a cosolvent, a chelating agent, an inert pellet, a coating agent, a lubricant, an anticaking agent and the like or their combinations of two or more.

15. The stable oral pharmaceutical formulation according to claim 14, wherein the preservative is in an amount of from about 0.05% to about 2% w/w, and the buffering agent is in an amount of from about 0.05% to about 10% w/w.

16. The stable oral pharmaceutical formulation according to claim 14, wherein the preservative is selected from the group consisting of erythorbic acid, fumaric acid, malic acid, sorbic acid, propyl gallate, ethyl oleate, methionine, monothioglycerol, thymol, tocopherols, sodium bisulfate, sodium metabisulfite, sodium sulfite, parabens (methylparaben, ethylparaben, propylparaben, butylparaben, isobutylparaben and the like) and/or their salts, benzoic acid, sodium benzoate, potassium sorbate, vanillin, benzyl alcohol, boric acid, calcium acetate, bentonite, cetrimide, chlorhexidine, cetylpyridinium chloride, cresol, chlorbutanol, magnesium trisilicate and the like or their combinations of two or more and the buffering agent is selected from the group consisting of sodium bicarbonate, potassium bicarbonate, magnesium hydroxide, magnesium lactate, magnesium gluconate, aluminum hydroxide, aluminum glycinate, citric acid, fumaric acid, tartaric acid, maleic acid, lactic acid, hydrochloric acid, phosphoric acid, glycine, glacial acetic acid, sodium acetate trihydrate, trisodium citrate, potassium chloride, hydroxymethyl aminomethane, sodium hydroxide, sodium citrate, sodium tartrate, sodium acetate, sodium carbonate, sodium phosphate monobasic, potassium phosphate, sodium polyphosphate, potassium polyphosphate, sodium pyrophosphate, potassium pyrophosphate, disodium hydrogen phosphate, dipotassium hydrogen phosphate, trisodium phosphate, tri potassium phosphate, potassium metaphosphate, magnesium oxide, magnesium carbonate, magnesium silicate, calcium acetate, calcium glycerophosphate, calcium chloride, calcium hydroxide, calcium lactate, calcium carbonate, calcium bicarbonate, and the like or their combinations of two or more.

17. The stable oral pharmaceutical formulation according to claim 1, further comprising an anticaking agent in an amount of from about 0.05% to about 10% w/w and selected from the group consisting of crospovidone, colloidal silicon dioxide, and the like or their combinations of two or more.

18. The stable oral pharmaceutical formulation according to claim 14, wherein the inert pellets are in an amount of from about 1% to about 15% w/w and are selected from the group consisting of mannitol, sugar, microcrystalline cellulose, vegetable gums, silicas, waxes and the like or their combinations of two or more, and the coating agent is in an amount of from about 0.5% to about 30% w/w and are selected from the group consisting of opadry, poly(vinyl alcohol), poly(vinyl pyrrolidone), hydroxy methyl cellulose, hydroxyethyl cellulose, hydroxypropyl methyl cellulose, hydroxy propyl cellulose, ethylcellulose, methyl cellulose, carboxymethyl cellulose, plasdone, eudragits, phthalates, cellulose acetate phthalate, hydroxypropyl methylcellulose phthalate, hydroxypropyl methylcellulose acetate, hydroxypropyl methylcellulose succinate, methacrylic and/or acrylic polymers, methacrylic acid copolymers, amino methacrylate copolymers, polydextrose, gum acacia and the like or their combinations of two or more.

19. A stable oral pharmaceutical formulation comprising:
an angiotensin converting enzyme inhibitor and/or a diuretic;
a sweetening agent; and
a pharmaceutically acceptable excipient,
wherein the angiotensin converting enzyme inhibitor is in an amount of from about 0.1% to about 2% w/w,
wherein the diuretic is in an amount of from about 0.15% to about 2.6% w/w, and
wherein the sweeting agent is in an amount of from about 0.5% to about 70% w/w.

20. A method of treating a patient in need of therapy for hypertension, coronary artery disease, heart failure, acute myocardial infarction and other related cardiovascular diseases, comprising administering a therapeutically effective amount of the stable oral pharmaceutical formulation according to claim 1.
